(19) United States
(12) Patent Application Publication
(10) Pub. No.: US 2026/0191647 A1
(43) Pub. Date: Jul. 9, 2026

(54) CARDIAC IMPLANTS COMPRISING SPIKED CELLS

(71) Applicant: Edwards Lifesciences Innovation (Israel) Ltd., Caesarea (IL)

(72) Inventors: Nikolai Gurovich, Hadera (IL); Michael Bukin, Pardes Hanna (IL)

(21) Appl. No.: 19/551,344

(22) Filed: Feb. 26, 2026

Related U.S. Application Data
(63) Continuation of application No. PCT/IB2024/055880, filed on Jun. 16, 2024.
(60) Provisional application No. 63/579,509, filed on Aug. 29, 2023.

(51) Int. Cl. A61F 2/24

(52) U.S. Cl. CPC A61F 2/2445; A61F 2/2466; A61F 2210/0014; A61F 2220/0016; A61F 2220/0025; A61F 2230/0006; A61F 2230/0069

(57) ABSTRACT

An implant is provided for use at a native cardiac valve of a subject. The implant has a central axis and a frame that, in an implanted configuration, defines a circumferential ring of spiked cells around the central axis. Each spiked cell has four corners consisting of an inner apex, two lateral corners connected, at respective intermediate junctions, to lateral corners of adjacent cells, and an outer apex oriented radially away from the central axis. The spiked cells are defined by struts that carry spikes. The intermediate junctions provide circumferential articulation such that each spiked cell bends with respect to adjacent cells to move its outer apex toward and away from the central axis independently of outer apices of the adjacent cells in a manner that maintains the spikes of that cell in tissue-gripping contact with tissue of the valve. Other embodiments are also described.

### CROSS-REFERENCES TO RELATED APPLICATIONS

The present application is a Continuation of International Patent Application PCT/IB2024/055880 to Gurovich et al., filed June 16, 2024, and titled "Annuloplasty devices comprising planar frames," which published as WO 2025/046319, and which claims priority to Provisional US Patent Application No. 63/579,509 to Gurovich et al., filed August 29, 2023, and titled "Annuloplasty devices comprising planar frames."

Each of the above documents is incorporated herein by reference in its entirety for all purposes.

### BACKGROUND

The heart is a muscular organ which pumps blood through the blood vessels of the circulatory system by contraction and expansion. In a healthy heart, blood flows in a single direction therethrough due to heart valves, which prevent backflow. During a normal heart contraction cycle, the heart valves open and close accordingly, while muscle heart tissues contracts. These muscle heart tissues can include various types of cavities and formations.

Ischemic heart disease can lead to valve regurgitation, such as mitral regurgitation. This is caused by the combination of weakened papillary muscles and dilation of the left ventricle, which displaces the papillary muscles and enlarges the annulus of the mitral valve. This prevents the leaflets from sealing properly when the valve is closed, resulting in blood flowing back from the left ventricle into the left atrium - a condition known as mitral regurgitation. This increases total stroke volume and decreases cardiac output, leading to the weakening of the left ventricle due to volume and pressure overload of the left atrium.

The same problem can occur in other parts of the heart and vascular system, and other valves. Annuloplasty, via implanting an annuloplasty ring or other device, can be used to improve leaflet coaptation by adjusting the shape of the native valve annulus. The use of percutaneous annuloplasty devices, such as transfemoral or transseptal, can be advantageous in certain scenarios.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In accordance with some implementations, there is provided an annuloplasty device comprising a planar frame configured to transition between a collapsed state and an expanded state. In some implementations, the planar frame comprises a plurality of spiked struts connected to each other at a plurality of junctions, a plurality of expandable segments and/or a plurality of spikes extending from the plurality of spiked struts.

In some implementations, the plurality of junctions comprises a plurality of outer apices and a plurality of inner apices. In some implementations, each expandable segment comprises at least two of the plurality of spiked struts, and is configured to transition between a collapsed state and an expanded state.

In some implementations, each two spiked struts of the plurality of spiked struts, which are connected to each other at a corresponding junction of the plurality of junctions, are configured to pivotably move relative to each other.

In some implementations, the plurality of spiked struts and the spikes extending therefrom are coplanar in the expanded state of the planar frame, such that the outer apices are positioned radially away from the inner apices in the expanded state of the planar frame.

In some implementations, each expandable segment defines a segmental length between two adjacent junctions of the plurality of junctions comprised in the expandable segment, wherein the two adjacent junctions defining the segmental length are at the same radial level relative to a central frame axis when the expandable segment is in an expanded state thereof.

In some implementations, the segmental length of each of the plurality of expandable segments is different between the collapsed state and the expanded state of the expandable segment.

In some implementations, each expandable segment further defines a segmental width which is perpendicular to the segmental length.

In some implementations, the planar frame is further configured to transition to a constricted state.

In some implementations, each of the plurality of expandable segments is further configured to transition to a constricted state.

In some implementations, the segmental width of each of the plurality of expandable segments is greater in the constricted state than in the expanded state of the expandable segment.

In some implementations, the annuloplasty device further comprises a tensioning member attached to the planar frame at an attachment end portion of the tensioning member.

In some implementations, the tensioning member extends from the attachment end portion along junctions of the plurality of junctions that define the plurality of segmental lengths.

In some implementations, the tensioning member is configured, upon being tensioned, to transition the planar frame to its constricted state.

In some implementations, the plurality of spikes is coplanar with the planar frame.

In some implementations, the plurality of spiked struts form at least one rung of spiked struts.

In some implementations, the at least one rung of spiked struts comprises at least two rungs of spiked struts.

In some implementations, the at least one rung of spiked struts comprises a single rung of spiked struts.

In some implementations, at least two of the plurality of expandable segments are attached, each, to a single other one of the plurality of expandable segments, while each of the other expandable segments are attached, each, to two the plurality of expandable segments.

In some implementations, the plurality of expandable segments comprises a first expandable segment which is coupled to an adjacent one of the expandable segments at one side thereof, and remains free ended without being coupled to any other expandable segment at an opposite side that included a first junction of the plurality of junctions.

In some implementations, each expandable segment of the plurality of expandable segments is configured to independently transition between its collapsed and expanded states.

In some implementations, the annuloplasty device further comprises a cylindrical frame coupled to the planar frame, wherein the cylindrical frame is configured to transition between a radially compressed configuration and a radially expanded configuration, and comprises a plurality of angled struts extending between a plurality of inflow junctions at an inflow end of the cylindrical frame, and a plurality of outflow junctions at an outflow end of the cylindrical frame.

In some implementations, the planar frame is coupled to the outflow end of the cylindrical frame.

In some implementations, the plurality of inner apices are coupled to the plurality of outflow junctions.

In some implementations, the plurality of outer apices are coupled to the plurality of outflow junctions.

In some implementations, the cylindrical frame is configured to flip from a state in which the inflow junctions are distal to the outflow junctions, to a state in which the inflow junctions are proximal to the outflow junctions.

In some implementations, the planar frame is coupled to the inflow end of the cylindrical frame.

In some implementations, the cylindrical frame tapers from the inflow end to the outflow end in its expanded configuration.

In some implementations, the annuloplasty device further comprises a strip coupled to the planar frame, the strip comprising a fabric base layer and a plurality of floating yarns extending therefrom.

In accordance with some implementations, a method (e.g., a method of treating a heart valve, a method of treating native anatomy, etc.) comprises navigating a delivery assembly that comprises an annuloplasty device having a planar frame, towards a native valve. In some implementations, the planar frame is retained in a collapsed state thereof inside an inner catheter of a delivery apparatus of the delivery assembly.

In some implementations, the method further comprises deploying the planar frame out of the inner catheter and engaging spiked struts of the planar frame with an annulus of the native valve, such that the spiked struts and spikes extending therefrom are coplanar with a plane defined by the annulus.

In some implementations, the method further comprises plicating tissue of annulus between adjacent spiked struts of a plurality of spiked struts of the planar frame.

In some implementations, each expandable segment of the plurality of expandable segments defines a segmental length between two adjacent junctions of the expandable segment, wherein the two adjacent junctions defining the segmental length are at the same radial level relative to a central frame axis when the expandable segment is in an expanded state thereof.

In some implementations, at least two of the plurality of expandable segments are attached, each, to a single other one of the plurality of expandable segments, while each of the other expandable segments are attached, each, to two the plurality of expandable segments.

In some implementations, the deploying the planar frame comprises sequentially deploying the expandable segments out of the inner catheter, over a curved path along the annulus.

In some implementations, the deploying the planar frame comprises uncovering the expandable segments by retracting the inner catheter.

In some implementations, the deploying the planar frame comprises uncovering the expandable segments by distally pushing a deployment plug attached to a deployment shaft of the delivery assembly, against the planar frame.

In some implementations, the deploying the planar frame out of the inner catheter further comprises expanding each of the plurality of expandable segments which is uncovered from the inner catheter.

In some implementations, the deploying the planar frame comprises placing each of the expandable segments, uncovered from the inner catheter, over the annulus such that the spikes engage with the annulus.

In some implementations, the expanding each of the plurality of expandable segments comprises approximating the junctions defining the segmental length of the corresponding expandable segment, thereby shortening the segmental length relative to the collapsed state of the same expandable segment.

In some implementations, the plicating the tissue of the annulus comprises the expanding the expandable segments.

In some implementations, the plicating the tissue of the annulus comprises proximally pulling a tensioning member attached to the planar frame at an attachment end portion of the tensioning member.

In some implementations, the pulling the tensioning member comprises transitioning at least one of the expandable segments to a constricted state, wherein the segmental length of the corresponding at least one expandable segment in the constricted state is less than the segmental length in its expanded state.

In some implementations, the method further comprising locking the tensioning member by a locker.

In some implementations, the locking the tensioning member further comprises trimming the tensioning member.

In some implementations, the annuloplasty device further comprises a cylindrical frame coupled to the planar frame, wherein the cylindrical frame is configured to transition between a radially compressed configuration and a radially expanded configuration.

In some implementations, the planar frame is configured to transition between an axially aligned state and a planar state.

In some implementations, the navigating the delivery assembly comprises retaining the cylindrical frame in the radially compressed configuration and retaining the planar frame in the aligned state inside the inner catheter.

In some implementations, the deploying the planar frame comprises at least partially expelling the annuloplasty device from the inner catheter such that the planar frame transitions to the planar state, while the cylindrical frame remains in the radially compressed configuration.

In some implementations, the navigating the delivery assembly comprises retaining the inflow junctions distal to the outflow junctions inside the inner catheter.

In some implementations, the deploying the planar frame comprises fully expelling the annuloplasty device from the inner catheter such that the cylindrical frame is flipped to position the inflow apices proximal to the outflow apices, and the planar frame transitions to the planar state.

In some implementations, the deploying the planar frame further comprises positioning the planar frame, in its planar state, over the annulus, such that the spikes engage with the annulus.

In some implementations, the at least partially expelling the annuloplasty device comprises retaining the cylindrical frame inside the inner catheter.

In some implementations, the deploying the planar frame further comprises expanding the cylindrical frame by uncovering the cylindrical frame from the inner catheter, thereby transitioning the planar frame to the expanded state.

In some implementations, the at least partially expelling the annuloplasty device comprises deploying the annuloplasty device out of the inner catheter, while the cylindrical frame is disposed in the radially compressed configuration, around a deflated inflatable balloon mounted on a balloon catheter of the delivery apparatus.

In some implementations, the transitioning the planar frame to the expanded state comprises distancing the junctions defining the segmental lengths of the corresponding expandable segments, thereby elongating the segmental lengths relative to the collapsed state of the planar frame.

Any of the above method(s) can be performed on a living subject (e.g., human or other animal) or on a simulation (e.g., a cadaver, cadaver heart, imaginary person, anthropomorphic ghost, simulator, such as a computer simulator, e.g., with the body parts, tissue, etc. being simulated). With a simulation, the body parts can optionally be referred to as "simulated" (e.g., simulated heart, simulated tissue, etc.) and can comprise, for example, computerized and/or physical representations.

A further understanding of the nature and advantages of the implementations are set forth in the following description and claims, particularly when considered in conjunction with the accompanying drawings. Other features and advantages will become apparent from the following detailed description and claims, particularly when considered with the accompanying drawings. The detailed description of implementations, as set out below to enable one to build and use the implementations, are not intended to limit the enumerated claims, but rather, they are intended to serve as examples of the claimed invention.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures:

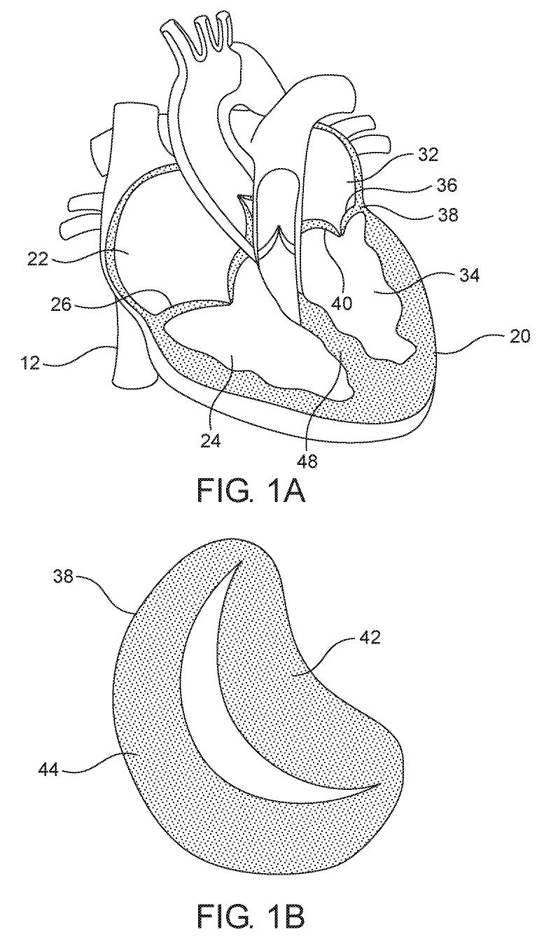

FIG. 1A shows a sectional view of a human heart.

FIG. 1B shows a mitral valve.

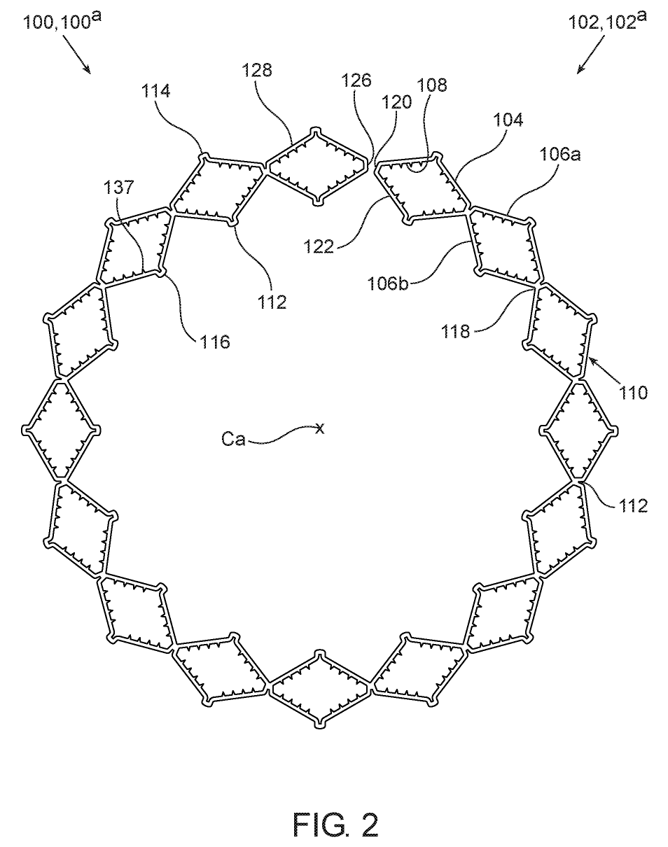

FIG. 2 shows an example planar frame that includes a single row of spiked cells.

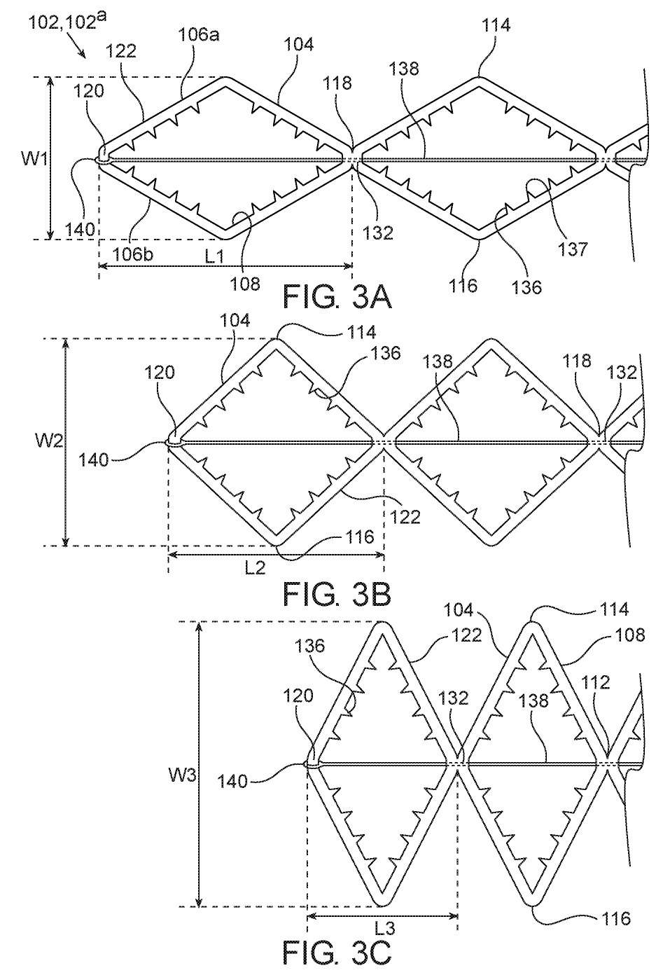

FIGS. 3A-3C illustrate different states of a portion of a planar frame.

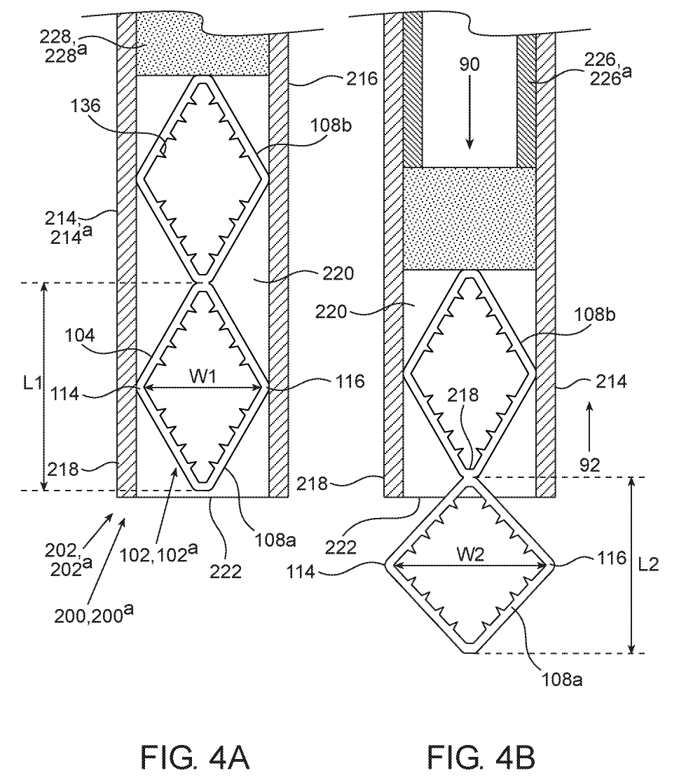

FIG. 4A shows a cross-sectional view of an example delivery apparatus, with spiked cells of a planar frame retained in a collapsed state inside an inner catheter of the delivery apparatus.

FIG. 4B shows a spiked cell deployed out of the inner catheter of FIG. 4A, in an expanded state of the uncovered spiked cell.

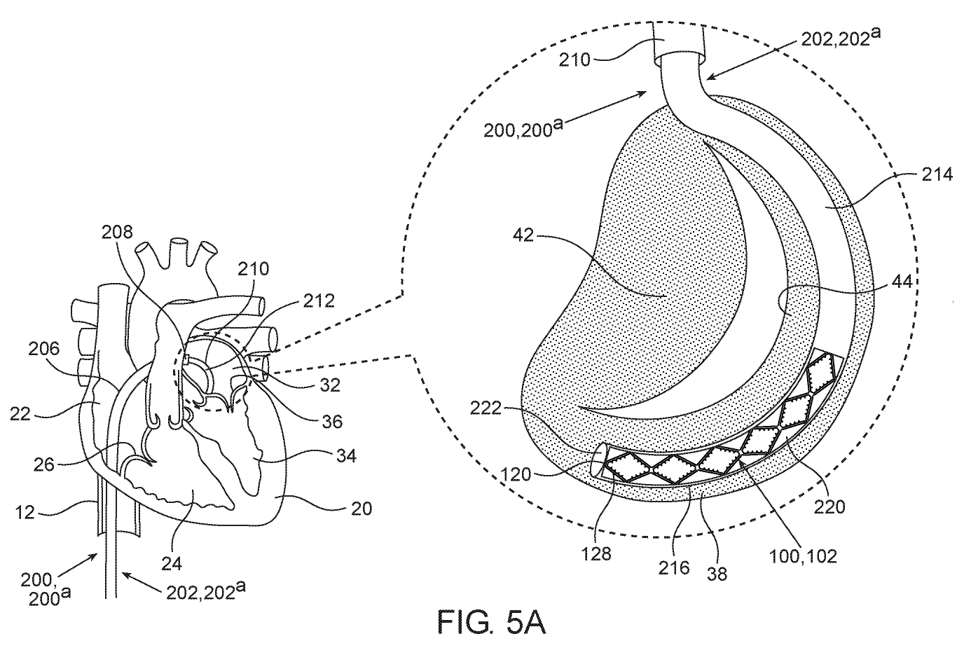

FIGS. 5A-5D show at least some phases of an example method of using an example delivery assembly to repair a native valve.

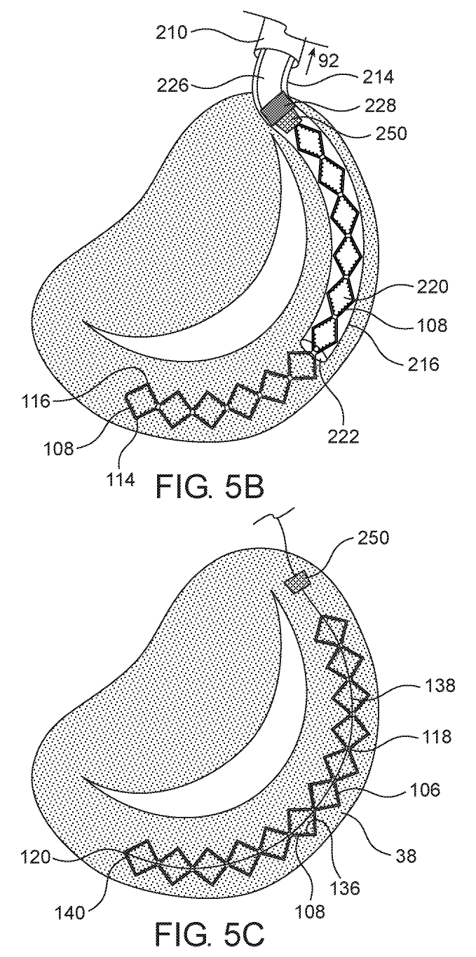

FIG. 6 shows an example planar frame that includes a single rung of spiked struts.

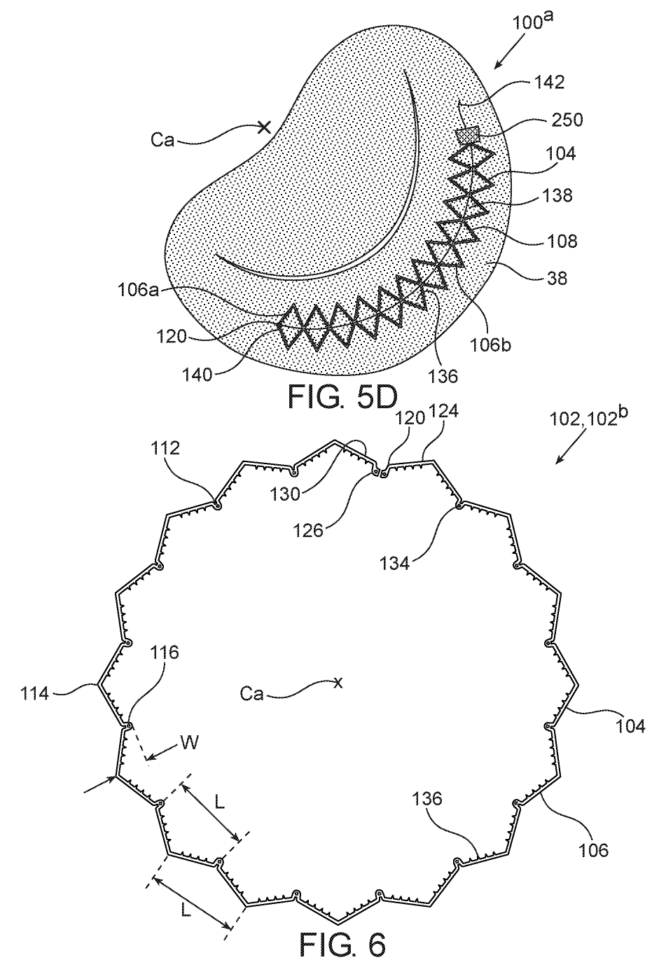

FIGS. 7A-7B show some phases of a method for implantation of an annuloplasty device that comprises the planar frame of FIG. 6.

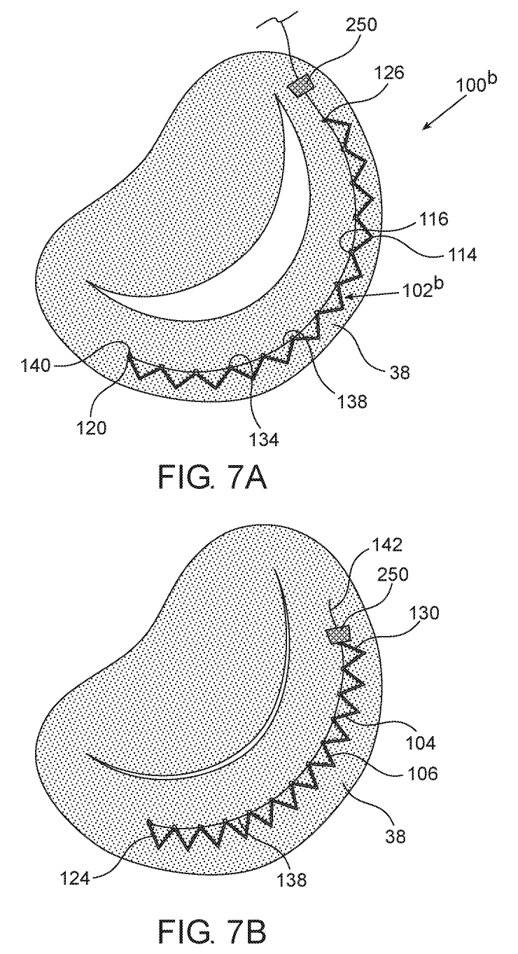

FIGS. 8A and 8B show a side view and a top view, respectively, of an example annuloplasty device that includes a planar frame attached to a cylindrical frame.

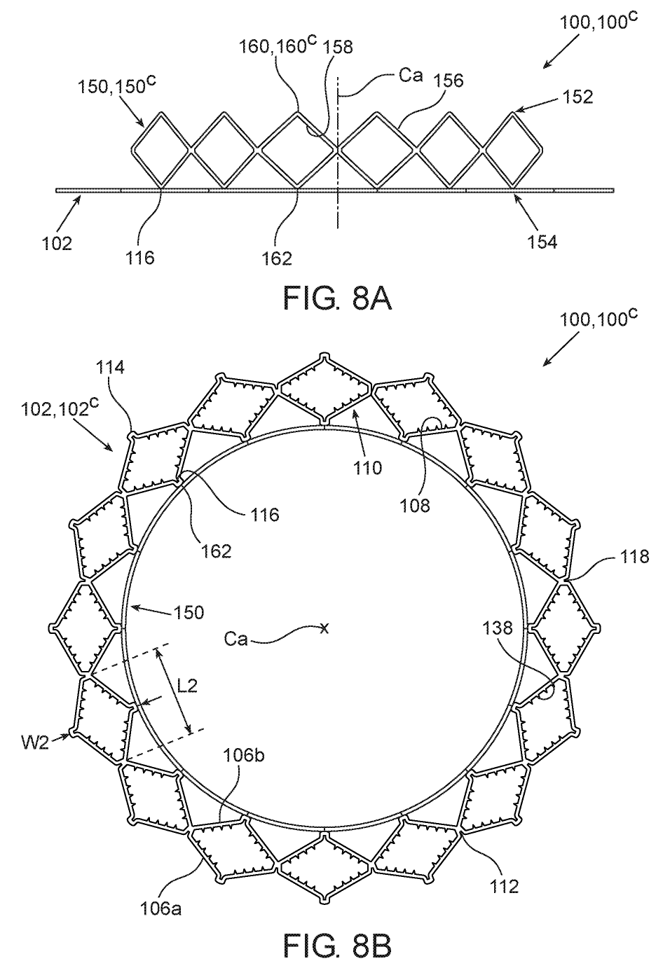

FIG. 9 shows an example annuloplasty device further comprising a tensioning member.

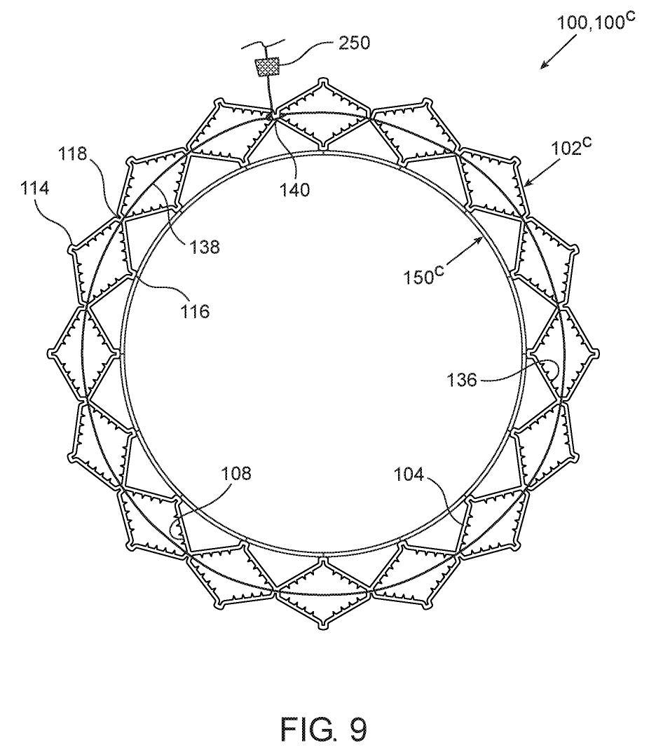

FIG. 10A shows a cross-sectional view of an example delivery apparatus, with the annuloplasty device of FIGS. 8A-8B residing inside an inner catheter of the delivery apparatus.

FIG. 10B shows the annuloplasty device partially expelled out of the inner catheter of FIG. 10A.

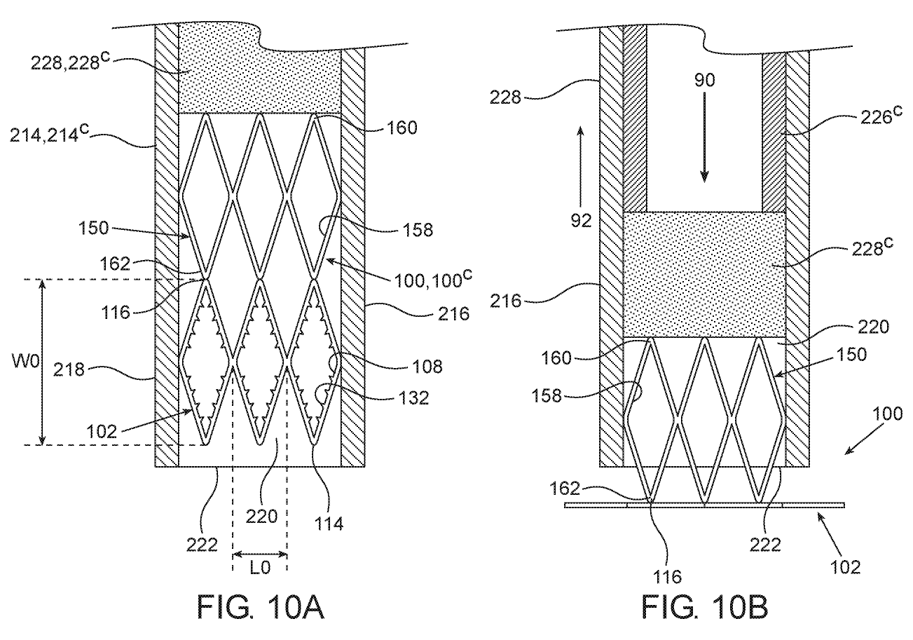

FIGS. 11A-11B show some phases of an example method of using delivery assembly to implant the annuloplasty device of FIGS. 8A-8B.

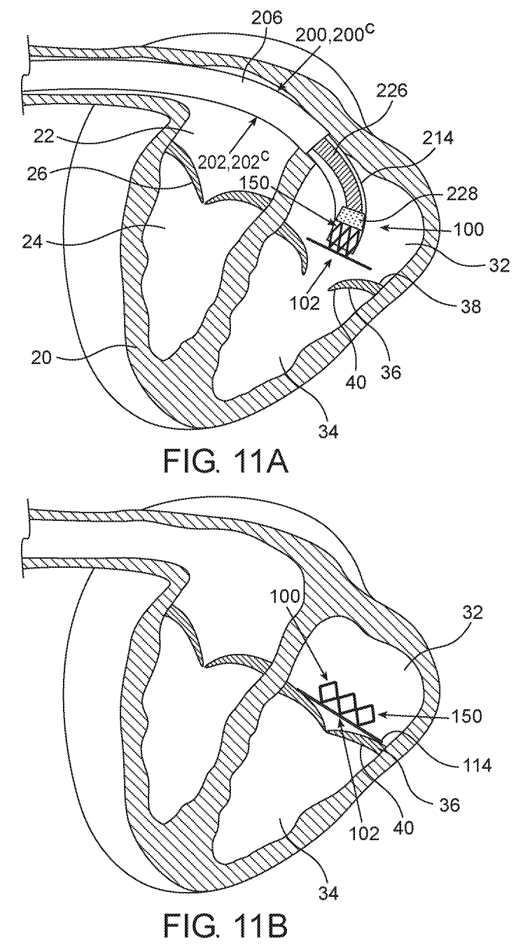

FIG. 12 shows an example annuloplasty device comprising a cylindrical frame equipped with a plurality of engaging struts.

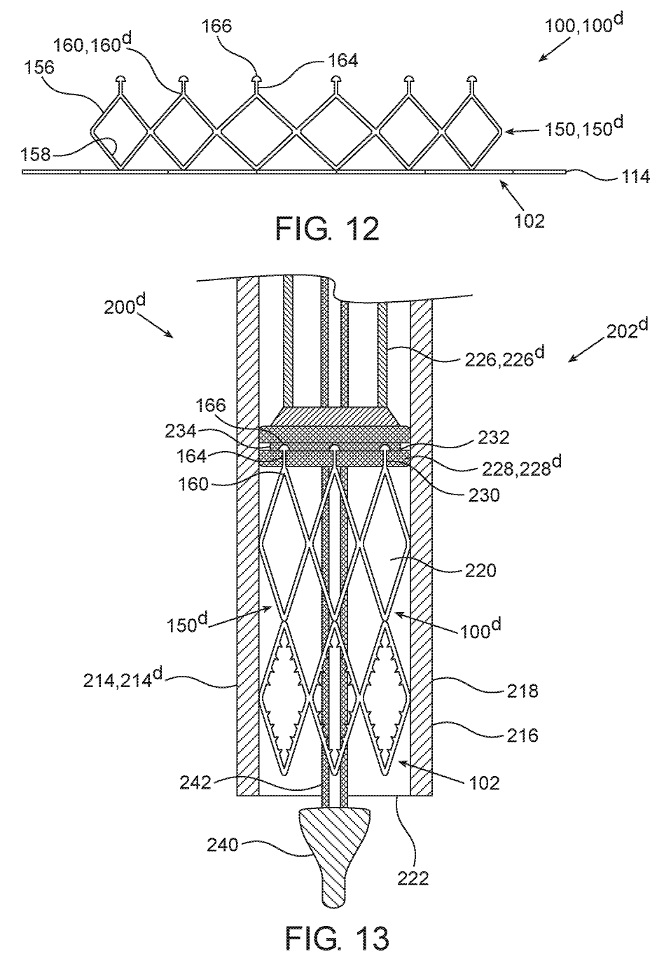

FIG. 13 shows a cross-sectional view of an example delivery apparatus comprising a deployment plug coupled to the cylindrical frame of the annuloplasty device of FIG. 12.

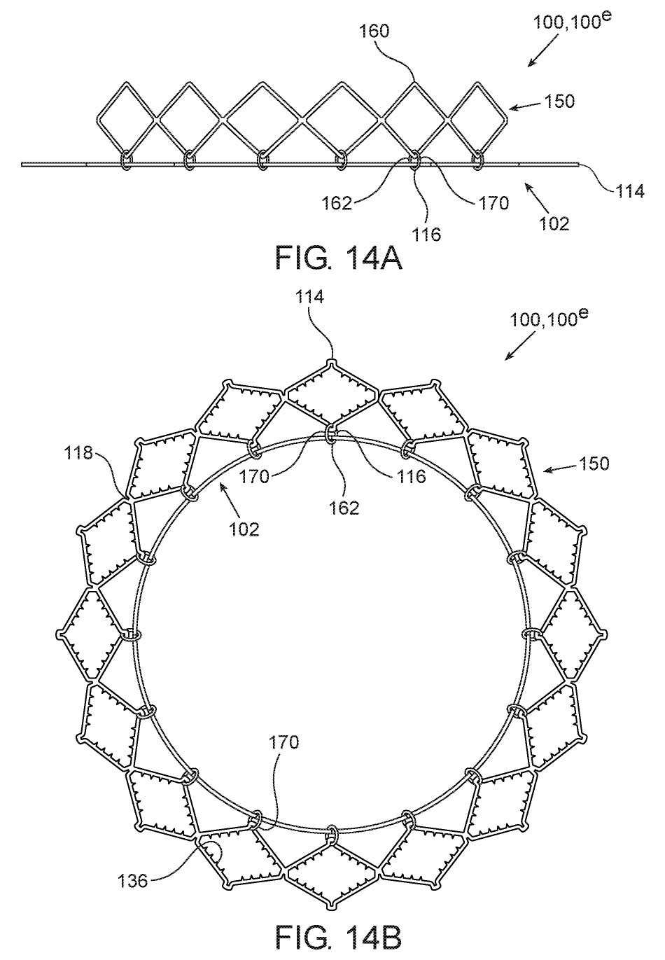

FIGS. 14A and 14B show a side view and a top view, respectively, of an example annuloplasty device that includes a planar frame coupled to a cylindrical frame by a plurality of connectors.

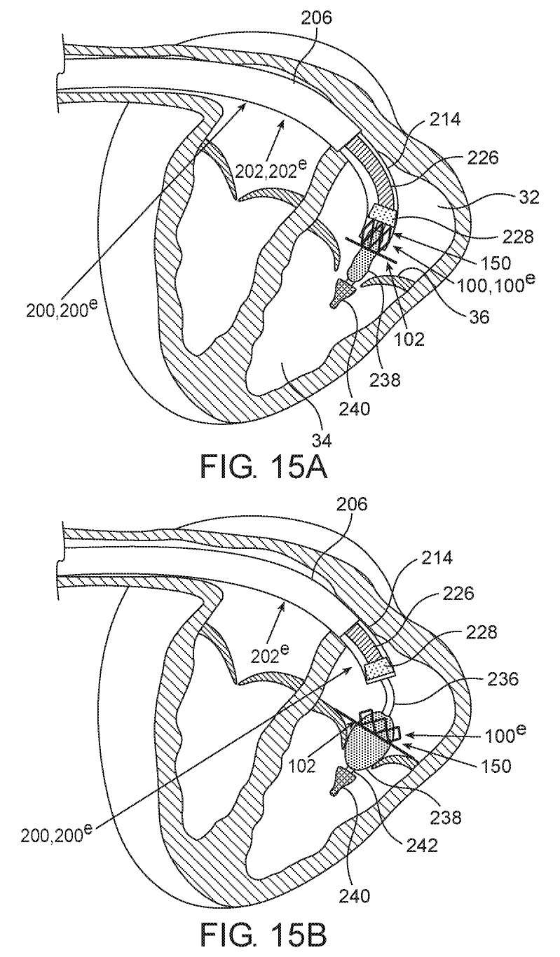

FIGS. 15A-15B show some phases of an example method of using a delivery assembly that includes an inflatable balloon mounted on a balloon catheter, to implant an annuloplasty device that includes a balloon-expandable cylindrical frame.

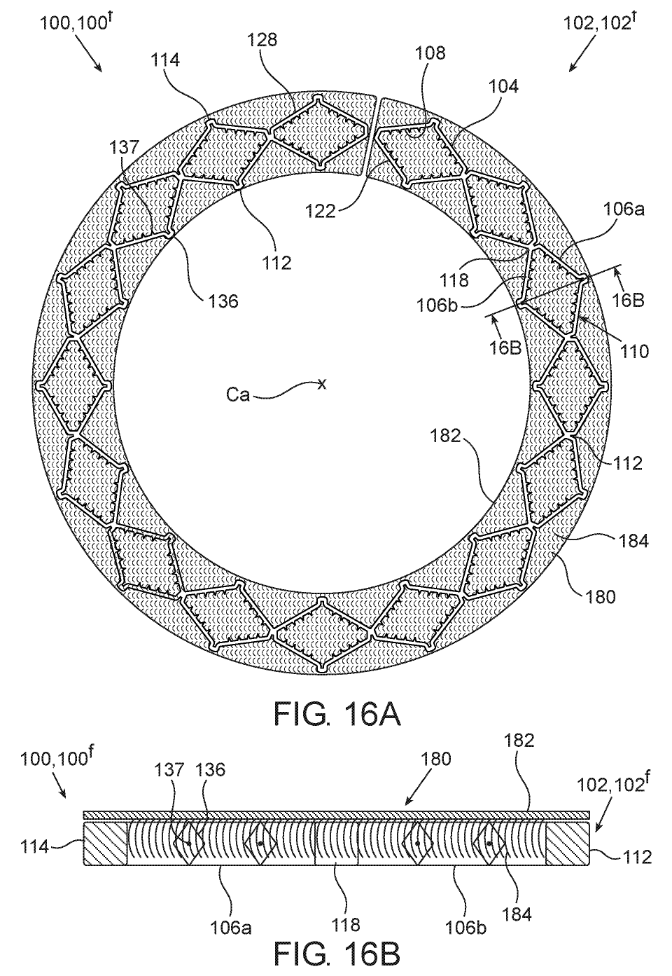

FIG. 16A shows a bottom view of an example annuloplasty device that includes a strip coupled to the annular frame.

FIG. 16B shows a cross-sectional view taken along line 16B-16B of FIG. 16A.

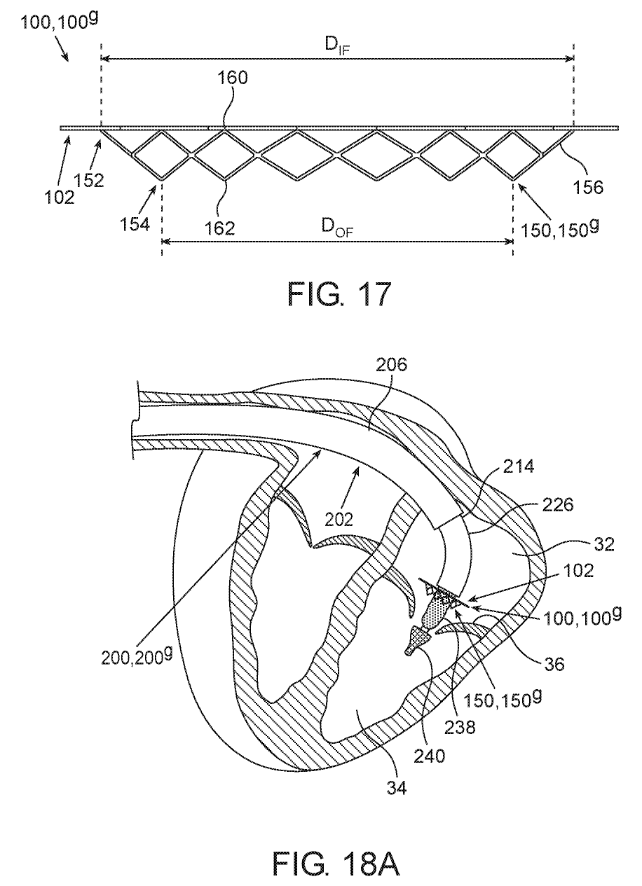

FIG. 17 shows a side view of an example annuloplasty device that includes a tapering cylindrical frame extending distally from the annular frame.

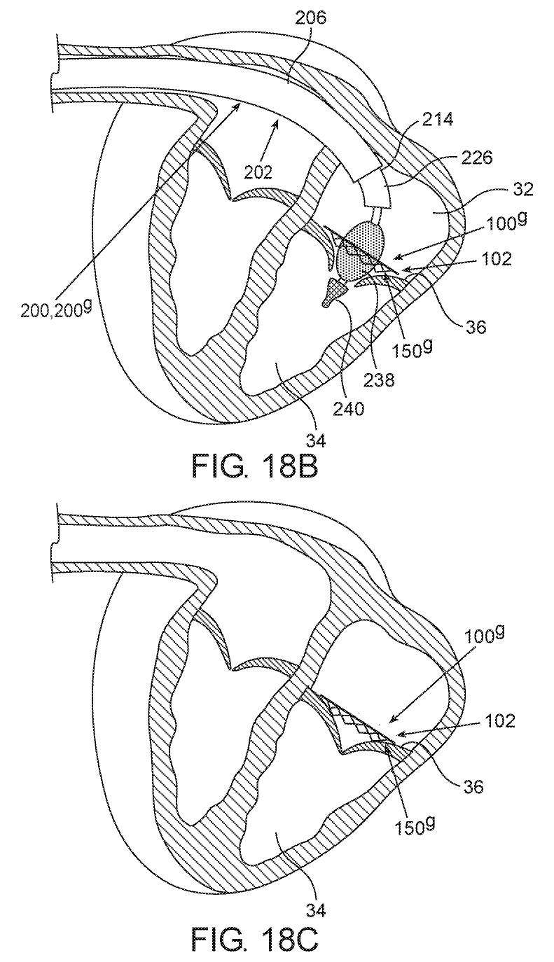

FIGS. 18A-18C show some phases of an example method of using a delivery assembly to implant the annuloplasty device of FIG. 17.

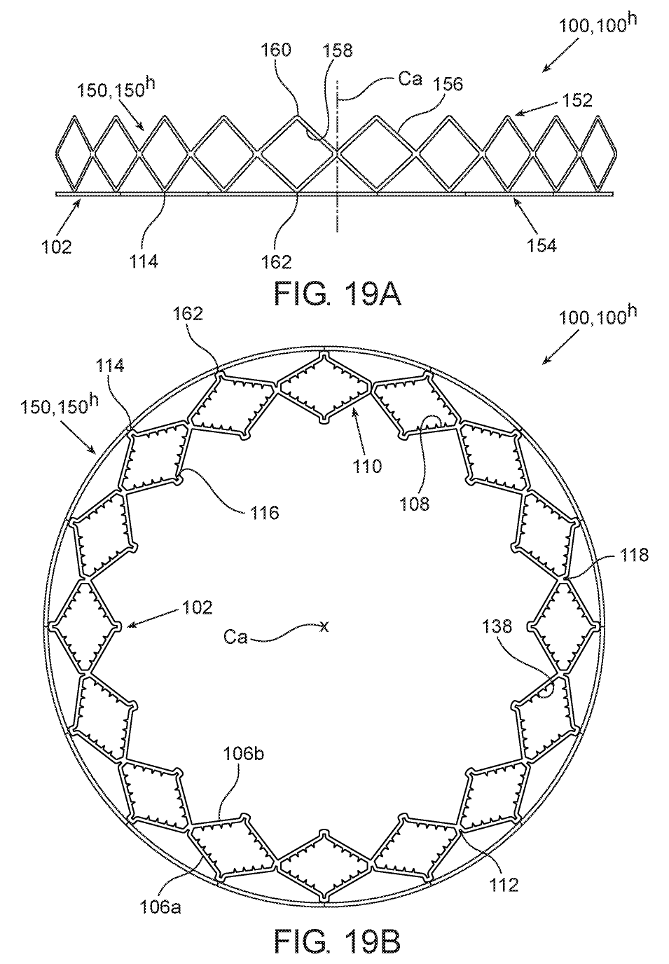

FIGS. 19A and 19B show a side view and a top view, respectively, of an example annuloplasty device that includes a cylindrical frame attached to outer junctions of the planar frame.

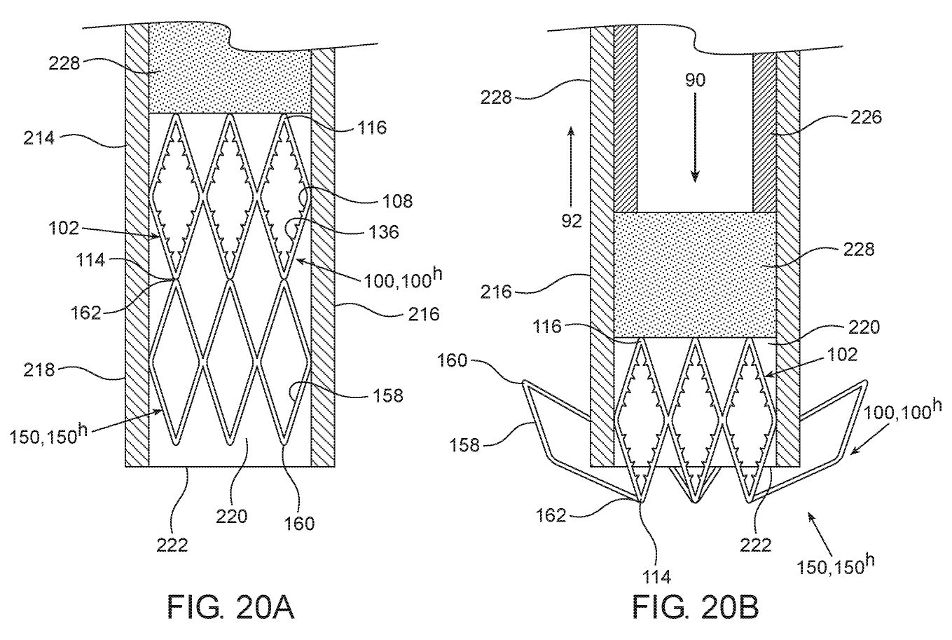

FIG. 20A shows a cross-sectional view of an example delivery apparatus, with the annuloplasty device of FIG. 19A-19B residing inside the inner catheter.

FIG. 20B shows the annuloplasty device partially expelled out of the inner catheter of FIG. 20A.

FIG. 20C shows the annuloplasty device fully expelled out of the inner catheter of FIGS. 20A-20B.

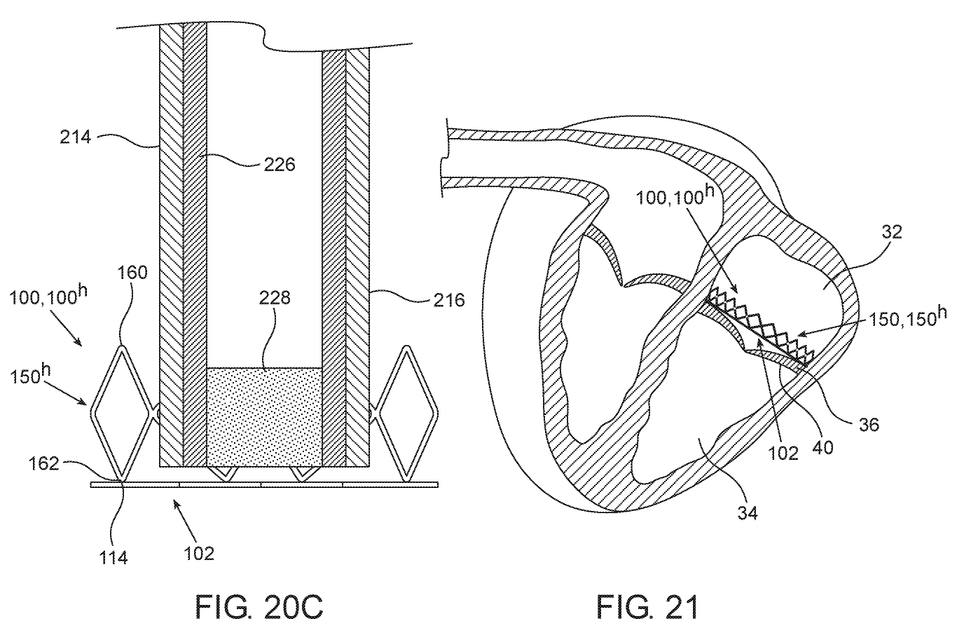

FIG. 21 shows the annuloplasty device of FIG. 19A-19B implanted in a patient's heart.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like "provide" or "achieve" to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms "a," "an," and "the" include the plural forms unless the context clearly dictates otherwise. Additionally, the terms "have" or "includes" means "comprises". Further, the terms "coupled", "connected", and "attached", as used herein, are interchangeable and generally mean physically, mechanically, chemically, magnetically, and/or electrically coupled or linked and does not exclude the presence of intermediate elements between the coupled or associated items absent specific contrary language. As used herein, "and/or" means "and" or "or", as well as "and" and "or".

Directions and other relative references may be used to facilitate discussion of the drawings and principles herein, but are not intended to be limiting. For example, certain terms may be used such as "inner," "outer," "upper," "lower," "inside," "outside,", "top," "bottom," "interior," "exterior," "left," right," and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an "upper" part can become a "lower" part simply by turning the object over. Nevertheless, it is still the same part and the object remains the same.

The term "plurality" or "plural" when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms "proximal" and "distal" are defined relative to the use position of a delivery apparatus. In general, the end of the delivery apparatus closest to the user of the apparatus is the proximal end, and the end of the delivery apparatus farthest from the user (e.g., the end that is inserted into a patient’s body) is the distal end. The term "proximal" when used with two spatially separated positions or parts of an object can be understood to mean closer to or oriented towards the proximal end of the delivery apparatus. The term "distal" when used with two spatially separated positions or parts of an object can be understood to mean closer to or oriented towards the distal end of the delivery apparatus. The terms "longitudinal" and "axial" are interchangeable, and refer to an axis extending in the proximal and distal directions, unless otherwise expressly defined.

The terms "axial direction," "radial direction," and "circumferential direction" have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms "integrally formed" and "unitary construction" refer to a construction that does not include any welds, fasteners, or other means for securing separately formed pieces of material to each other.

As used herein, operations that occur "simultaneously" or "concurrently" occur generally at the same time as one another, although delays in the occurrence of operation relative to the other due to, for example, spacing between components, are expressly within the scope of the above terms, absent specific contrary language.

As used herein, terms such as "first," "second," and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term "substantially" means the listed value and/or property and any value and/or property that is at least 75% of the listed value and/or property. Equivalently, the term "substantially" means the listed value and/or property and any value and/or property that differs from the listed value and/or property by at most 25%. For example, "at least substantially parallel" refers to directions that are fully parallel, and to directions that diverge by up to 22.5 degrees.

In the present disclosure, a reference numeral that includes an alphabetic label (for example, "a," "b," "c," etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting annuloplasty devices and modifying native valves in a patient’s heart. In some implementations, the native valve can be a mitral valve or a tricuspid valve. The modification can reduce a length of the annulus around native leaflets of the native valve, so as to improve coaptation between the leaflets when the valve is closed. While described with respect to a mitral valve, it should be understood that the disclosed examples can be adapted to deliver and utilize annuloplasty devices that can modify other native annuluses of the heart (e.g., the aortic, pulmonary, and tricuspid annuluses) or any other orifice in a patient body (e.g., the left atrial appendage), and can be used with any of various delivery approaches (e.g., retrograde, antegrade, transseptal, transventricular, transatrial, transapical, etc.).

FIG. 1A shows a sectional view of a human heart 20. The heart has a four-chambered conical structure that includes the right atrium 22, the right ventricle 24, the left atrium 32 and the left ventricle 34. The wall separating between the left and right sides of the heart is referred to as the septum 48. The native mitral valve 36 is positioned between the left atrium 32 and the left ventricle 34. The native tricuspid valve 26 is positioned between the right atrium 22 and the right ventricle 24.

During the diastolic phase, or diastole, deoxygenated blood flows from the right atrium 22 into the right ventricle 24 through the tricuspid valve 26. During systole, leaflets of a normally functioning tricuspid valve 26 close to prevent the venous blood from regurgitating back into the right atrium 22. When the tricuspid valve 26 does not operate normally, blood can backflow or regurgitate into the right atrium 22.

The native mitral valve 36 comprises a mitral annulus 38 and a pair of mitral leaflets 40 extending downward from the annulus 38. The leaflets 40 of the mitral valve 36 include an anterior leaflet 42 and a posterior leaflet 44, shown in FIG. 1B. When operating properly, the anterior leaflet 42 and the posterior leaflet 44 function together as a one-way valve to allow blood flow from the left atrium 32 to the left ventricle 34. Specifically, during diastole, when the muscles of the left atrium 32 and the left ventricle 34 dilate, oxygenated blood flows from the left atrium 32, through the mitral valve 36, into the left ventricle 34. During systole, when the muscles of the left atrium 32 relax and the left ventricle 34 contacts, the blood pressure within the left ventricle 34 increases so as to urge to two mitral leaflets 40 to coapt, thereby preventing blood flow from the left ventricle 34 back to the left atrium 32.

Valvular heart disease can affect functioning of the mitral valve 36, as well other heart valves, including the tricuspid valve 26. Mitral regurgitation occurs when the native mitral valve 36 fails to close properly and blood flows back into the left atrium 32 from the left ventricle 34 during systole. Possible causes of this include leaflet prolapse, dysfunctional papillary muscles, issues with chordae tendineae, and/or stretching of the mitral valve annulus due to left ventricle dilation. Tricuspid regurgitation occurs when the tricuspid valve 26 fails to close properly and blood flows back into the right atrium 22 from the right ventricle 24.

FIG. 2 shows an example annuloplasty device 100 that includes a planar frame 102. The planar frame 102 is movable between a collapsed state, and an expanded state. In some implementations, the planar frame 102 is movable between a collapsed state, an expanded or free state, and a contracted state. In some implementations, the planar frame 102 is movable between a collapsed state and a contracted state, wherein an expanded state can be defined as a transitional state between the collapsed and contracted states.

In some implementations, annuloplasty device 100 comprises a plurality of spiked struts 104 intersecting at junctions 112, arranged to form at least one rung 106 of spiked struts 104. In some implementations, the planar frame 102 further comprises a plurality of spikes 136 extending from the spikes struts 104, configured to engage (and in some instances penetrate) a native tissue, such as that of annulus 38 and/or portions of native leaflets 40.

“Spiked struts,” “spiked cells,” ”spikes,” etc. as used herein can refer to a wide variety of projections associated with a strut, stent, apparatus, etc. and can be configured in a variety of ways and shapes, even if different from the example spikes shown in the figures. In some implementations, the spikes can be configured as barbs, hooks, teeth, ridges, triangles, rectangles, semicircles, arches, jagged edges of strut or other component, or another type of friction-enhancing element. In some implementations, the “spiked struts” can be configured as barbed struts, friction-enhanced struts, jagged struts, projection struts, hooked struts, pincer struts, etc. In some implementations, the spikes (e.g., barbs, hooks, teeth, friction-enhancing elements, projections, edges, etc.) can be all of the same or similar size and/or configuration, or they can have varying sizes and/or configurations (e.g., all different configurations, some of one configuration and some of another configuration, etc.).

In some implementations, each spike 136 terminates with a sharp tip 137.

At least some of the spiked struts 104 can be pivotable or bendable relative to each other, so as to permit expansion or compression of planar frame 102. For example, the planar frame 102 can be formed from a single piece of material, such as a metal sheet, via various processes such as, but not limited to, laser cutting, electroforming, and/or physical vapor deposition, while retaining the ability to collapse/expand radially in the absence of hinges and like.

Various example implementations for annuloplasty devices 100, planar frames 102, and/or components thereof, can be referred to, throughout the specification, with superscripts, for ease of explanation of features that refer to such example implementations. It is to be understood, however, that any reference to structural or functional features of any device or component, without a superscript, refers to these features being commonly shared by all specific example implementations that can be also indicated by superscripts. In contrast, features emphasized with respect to an example implementation of any device or component, including annuloplasty device 100 and/or planar frames 102 thereof, referred to with a superscript, may be optionally shared by some but not necessarily all other example implementations. For example, planar frame 102^{a} of annuloplasty device 100^{a} illustrated in FIG. 2 is an example implementation of planar frame 102, and thus can include some or all of the features described for planar frame 102 throughout the current disclosure, except that while a planar frame 102 can generally include one or more strut rungs 106, the example frame 102^{a} includes two rungs 106a and 106b, together defining a single row 110 of spiked cells 108.

FIG. 2 shows example planar frame 102^{a} in an expanded state, such that the rungs 106 of spiked struts 104 and/or row 110 of spiked cells 108 are circumferentially disposed around a central frame axis Ca. Central frame axis Ca is an axis around which the planar frame 102 is disposed when implanted in a patient's body. For example, when an annuloplasty device 100 that includes planar frame 102 is implanted at a mitral valve 36, the central frame axis Ca can be directed from the left atrium 32 to the left ventricle 34.

As will be further described below, a planar frame 102 can be deployed and expanded to extend along an arcuate path that does not necessarily enclose a full circle, in which case the central frame axis Ca can be defined as the center of curvature of a circular arc that best approximates the curve along which the planar frame 102 extends between a first junction 120 and a final junction 126 (described in greater detail below).

The term "planar frame", as used herein, refers to a frame configured which extends, in an expanded state and/or contracted state thereof, along a plane which is substantially orthogonal to the central frame axis Ca. A planar frame is a frame in which all spiked struts (or otherwise configured struts) are arranged on the same plane, at least when the entire planar frame 102 is in an expanded state, such that the outer apices 114 are positioned radially away from the inner apices 116 along a shared plane, relative to the central frame axis. For example, a planar frame 102 configured for implantation at a mitral annulus, can assume an expanded state and/or contracted state that is substantially parallel to, or co-planar with, a plane defined by the annulus 38. The term "planar" can mean perfectly planar or planar with spiked struts 104 deviating from perfectly planar by up to 5°, 10°, 15°, or 20°.

In some implementations, each spiked cell 108 can be a diamond-shaped cell defined by four spiked struts 104. While FIG. 2 illustrates a planar frame 102^{a} that includes two rungs 106 of struts, together forming a single row 110 of cells, it is to be understood that this is shown by way of illustration and not limitation, and that planar frame 102 can include any number of strut rungs 106 and any number of cell rows 110.

In some implementations, junctions 112 comprise outer apices 114 oriented away from the central frame axis Ca, and inner apices 116 oriented towards the central frame axis Ca, such that the inner apices 116 are closer to the central frame axis Ca than the outer apices 114. In some implementations, junctions 112 further comprise intermediate junctions 118, disposed radially between, and circumferentially offset from, the outer and inner apices 114, 116.

Intermediate junctions 118 can be disposed between adjacent spiked cells 108, such that at least some of the spiked cells 108 are interconnected by intermediate junctions 118. As shown in FIG. 2, frame 102 can be curved around the central frame axis Ca in its expanded and/or contracted state. In some implementations, adjacent spiked cells 108 can be pivotable or bendable relative to each other around intermediate junctions 118, such that each spiked cell 108 can assume a different orientation with respect to the central frame axis Ca. Planar frames 102 that include a single row 110 of cells, as shown for planar frame 102^{a}, or a single rung 106 of spiked struts 104, as will be described below with respect to planar frame 102^{b} illustrated in FIG. 6, may be better suited to allow such pivotable or bendable motion between adjacent segments of the frames.

In some implementations, planar frame 102 includes at least two expandable segments, such as spiked cells 108, each of which is connected to an adjacent cell on one end, but remains unconnected to any other cell on the opposite intermediate junction. For example, planar frame 102^{a} is illustrated in FIG. 2 to include a first cell 122 attached to an adjacent cell by an intermediate junction on one lateral end thereof, while an opposite intermediate junction 118, which can be also referred to as a first junction 120, remains free ended and unattached to any other spiked cell 108. Example planar frame 102^{a} is also shown to include a final cell 128 attached to a preceding spiked cell 108 by an intermediate junction, but remaining free ended and unattached to any other cell at an opposite intermediate junction 118, which can be also referred to as a final junction 126.

In some implementations, a cell row 110 can include a first cell 122 free ended at a first junction 120 and attached to a subsequent spiked cell 108 at an intermediate junction 118 opposite to the first junction 120, and a final cell 128 free ended at a final junction 126 and attached to a preceding spiked cell 108 at an intermediate junction 118 opposite to the final junction 126. In some implementations, each of the spiked cells 108 disposed between the first cell 122 and the final cell 128 is attached to two adjacent spiked cells 108 at its opposite lateral ends.

While the planar frame 102^{a} is illustrated in FIG. 2 to assume a substantially circular configuration, spanning almost 360° around the central frame axis Ca in the expanded and/or contracted state, it is to be understood that this is shown by way of illustration and not limitation, and that any other curved but not necessarily circular shape is contemplated, such as an arc shown in FIGS. 5C-5D for example.

In some implementations, expandable segments of a planar frame 102 can independently transition between collapsed and expanded states, irrespective of adjacent portions of the frame 102. For example, in a planar frame 102^{a} that includes a row 110 of spiked cells 108, each spiked cell 108 can transition between collapsed and expanded states thereof, while adjacent spiked cell(s) 108 can transition to or remain in a different state. In some implementations, each expandable segment of a planar frame, such as a spiked cell 108, is movable between a collapsed state, and an expanded state. In some implementations, each expandable segment of a planar frame, such as a spiked cell 108, is movable between a collapsed state, an expanded or free state, and a contracted state.

In some implementations, each expandable segment of a planar frame, such as a spiked cell 108, is movable between a collapsed state and a contracted state, wherein an expanded state can be defined as a transitional state between the collapsed and contracted states.

When a planar frame 102 that includes a plurality of expandable segments that can independently move between various states, such as spiked cell 108 that can independently move between collapsed and expanded states, and/or between collapsed and constricted states, the planar frame 102 is termed to be in an expanded state, as a whole, when each of its extendable segments, such as each of the spiked cells 108, is in an expanded state. The planar frame 102, in such cases, is similarly termed to be in a compacted state, as a whole, when each of its extendable segments, such as each of the spiked cells 108, is in a compacted state. The planar frame 102, in such cases, is similarly termed to be in a constricted state, as a whole, when each of its extendable segments, such as each of the spiked cells 108, is in a constricted state.

A collapsed state and an expanded state of any expandable segment of a planar frame 102, differ from each other by a different segmental length between two adjacent junctions along the same level of the expandable segment. For example, transitioning of any spiked cell 108 between collapsed and expanded states thereof, means that the distance between its opposing intermediate junctions 118 changes during such transitioning, thereby either shortening or elongating the segmental length L. Transitioning to a constricted state of the expandable segment similarly refers to further change in segmental length L. For example, transitioning of any spiked cell 108 from an expanded state to a constricted state means that the segmental length L between its opposing intermediate junctions 118 gets shorter.

In some implementations, such as in the case of example planar frame 102^{a} shown in FIG. 2, or such as in the case of example planar frame 102^{a} that will be described in greater detail below with respect to FIG. 6, each expandable segment can independently transition from a collapsed state to an expanded state, having a longer segmental length L in the collapsed state than in its expanded state. In some implementations, such as in the case of example planar frame 102^{c} that will be described in greater detail below with respect to FIGS. 8A-8B, the entire planar frame can transition from a collapsed state to an expanded state, forcing all of its expandable segments to simultaneously transition therewith (that is to say, without allowing independent transition of one expandable segment while other expandable segments remain in a different state), and the segmental length L of any of its expandable segments is shorter than in the expanded state.

FIGS. 3A-3C illustrate different states of a portion of a planar frame 102, shown by way of example for two distal spiked cells 108 of planar frame 102^{a}. FIG. 3A shows spiked cells 108 of a planar frame 102 in a collapsed state. FIG. 3B shows the spiked cells 108 in an expanded state. FIG. 3C shows the spiked cells 108 in a contracted state. The distance between two adjacent junctions 112 that lie on the same level, is defined as the segmental length L. The term "lie on the same level" refers to junctions that are at the same radial distance from central frame axis Ca, at least when the planar frame 102 is in the expanded state, such as upon being implanted. For example, in some implementations, all outer apices 114 are at the same level relative to each other, all the inner apices 116 are at the same level relative to each other, and all intermediate junctions 118 are at the same level relative to each other.

Each spiked cell 108 can define a segmental length L between adjacent intermediate junctions 118 of the cells, and define a segmental width W between the outer apex 114 and the inner apex 116 of the spiked cell 108.

In the collapsed state of a segment of the frame 102, such as a spiked cell 108 of the frame 102^{a} shown in FIG. 3A, the segmental length L1 can be greater than the segmental width W1. In the expanded state, shown in FIG. 3B, the two opposing intermediate junctions 118 of the segment (such as spiked cell 108) move closer to each other, such that the segmental length L2 in the expanded state is less than the segmental length L1 in the collapsed state. When the expanded segment of the planar frame 102 is a spiked cell 108, its outer apex 114 and inner apex 116 move away from each other in the expanded state, such that the segmental width W2 in the expanded state is greater than the segmental width W1 in the collapsed state.

In the contracted state, shown in FIG. 3C, the two opposing intermediate junctions 118 of the segment (such as spiked cell 108) move further towards each other, such that the segmental length L3 in the contracted state is less than the segmental length L2 in the expanded state. When the expanded segment of the planar frame 102 is a spiked cell 108, its outer apex 114 and inner apex 116 move farther away from each other in the contracted state, such that the segmental width W3 in the contracted state is greater than the segmental width W2 in the expanded state.

In some implementations, annuloplasty device 100 can further comprise a tensioning member 138 attached at an attachment end portion 140 thereof to an end of the planar frame 102, such as to the first junction 120. Tensioning member 138 can be in the form of a string, a suture, a wire, a cable, and the like. Attachment end portion 140 can be in the form of a loop or a knot tied around a junction 112, such as the first junction 120, as illustrated in FIG. 3A-3C, though any other manner of attaching the tensioning member 138 to a junction 120 is contemplated. During delivery, the tensioning member 138 can extend proximally from the attachment end portion 140 at the first junction 120, such as towards a handle (not shown) of a delivery apparatus configured to deliver the annuloplasty device to the site of implantation.

In some implementations, tensioning member 138 extends along a series of subsequent junctions 112 that define segmental lengths L therebetween, such as subsequent intermediate junctions 118. In some implementations, at least some of the plurality of junctions 112 include guiding structures along which and/or through which the tensioning member 138 can extend. In some implementations, at least some of the junctions 112, such as intermediate junctions 118 shown in FIGS. 3A-3C, include channels 132 through which the tensioning member 138 can extend. In the example implementation illustrated in FIGS. 3A-3C, all or most of the intermediate junctions 118, excluding the first junction 120 and the final junction 126, can include such channels 132 through which the tensioning member 138 can proximally extend from the attachment end portion 140 at first junction 120.

It is to be understood that in some implementations, tensioning member 138 can be coupled to junctions 112 of a planar frame 102, including to intermediate junctions, by other suitable means. In some implementations, the tensioning member 138 can be slidable along one or more junctions 112, in a manner that allows it to change in length between two junctions 112 it is coupled to. For example, a planar frame 102 can be devoid of channels 132, and a tensioning member 138 can be looped around intermediate junctions 118.

In some implementations, when the tensioning member 138 is proximally pulled, it causes the junctions 112 to which it is attached and/or along or through which it passes, to move closer to each other. For example, tensioning member 138 can be proximally pulled to facilitate transitioning of one or more expandable segments of the frame 102, such as spiked cells 108 of planar frame 102^{a}, from one state, such as the expanded state shown in FIG. 3B, to another state, such as the contracted state shown in FIG. 3C.

FIGS. 4A-4B show cross-sectional views of a distal portion of an example delivery assembly 200 that includes an example delivery apparatus 202 adapted to deliver an annuloplasty device 100, such as annuloplasty device 100 described above with respect to FIGS. 2-3C.

In some implementations, delivery apparatus 202 can include an inner catheter 214 defining an inner catheter lumen 220 in which an annuloplasty device 100 can reside, for example in a collapsed state thereof, during delivery towards a site of implantation. In some implementations, the inner catheter 214 includes a distal end portion 216 that terminates at a distal opening 222.

In some implementations, the distal end portion 216 of can comprise a capsule 218 that can be provided as a separate component that can be attached to a distal end of a tubular portion that forms the remainder of the inner catheter 214. In some implementations, capsule 218 can be integrally formed with the remainder of the inner catheter 214, such that the distal end portion 216, defined as an integral distal portion of the tube forming the inner catheter 214, functions as a capsule 218.

A planar frame 102^{a} that includes only two spiked cells 108 is shown in FIGS. 4A-4B for ease of illustration, and it is to be understood that a planar frame 102 can include more than two expandable segments, such as more than two spiked cells 108 shown in FIGS. 4A-4B. Moreover, while not shown in FIG. 4A-4B, it is to be understood that the annuloplasty device can further include a tensioning member 138.

FIG. 4A shows an example delivery configuration, in which the entire annuloplasty device 100, including the entire planar frame 102, resides inside the inner catheter lumen 220, proximal to the distal opening 222. For example, the frame 102 is positioned, in FIG. 4A, such that its first junction 120, which is the distal-most junction 112, is at the level of, or proximal to, the distal opening. The diameter of inner catheter lumen 220 can be similar to the segmental width W1 in the collapsed state, forcing all segments of the planar frame 102 to retain a collapsed state while positioned inside inner catheter 214. In this state, the delivery assembly 200 can be navigated towards the site of implantation.

In some implementations, the delivery apparatus 202 can include a deployment shaft 226 equipped with a deployment plug 228 attached to a distal end thereof, coaxially located within the inner catheter 214. In some implementations, the deployment shaft 226 extends proximally from the deployment plug 228, such as towards a handle (not shown) of the delivery apparatus, and can be configured to facilitate deployment of the annuloplasty device 100 from inside the distal end portion 216 of the inner catheter 214. Deployment shaft 226 and inner catheter 214 can be axially movable relative to each other.

FIGS. 4A-4B shows an example delivery assembly 200^{a} comprising an example delivery apparatus 202^{a}. Delivery apparatus 202^{a} is an example implementation of delivery apparatus 202, and thus can includes some or all of the features described for delivery apparatus 202 throughout the current disclosure, except that delivery apparatus 202^{a} includes a deployment shaft 226 that can be in the form of a push shaft 226^{a}, equipped with a deployment plug 228 that can serve as a push member 228^{a}.

In some implementations, prior to deployment of the annuloplasty device 100, as shown for example in FIG. 4A, the push member 228^{a} can be positioned proximal to the annuloplasty device 100. FIG. 4B shows the delivery assembly 200^{a} during deployment, with at least once expandable segment, such as a spiked cell 108a of planar frame 102^{a}, deployed out of the inner catheter 214, while at least one other expandable segment, such as another spiked cell 108b, still resides, in a collapsed undeployed state, inside the inner catheter 214.

In some implementations, push shaft 226^{a} is pushed in a distal direction 90, while the inner catheter 214^{a} can remain stationary (i.e., axially immovable), to move at least a portion of the planar frame 102 out of the inner catheter 214^{a}. In some implementations, the inner catheter 214^{a} is pulled in the proximal direction 92, while the push shaft 226^{a} can remain stationary (i.e., axially immovable) with the push member 228^{a} pressed against the proximal end of the planar frame 102 (such as against the final junction 126, which is the proximal-most junction 112) to provide a counterforce against the frame 102, such that at least a portion of the planar frame 102 is exposed out of the inner catheter 214^{a}. In some implementations, the push shaft 226^{a} can be distally pushed while the inner catheter 214^{a} can be simultaneously proximally pulled.

As shown in FIG. 4B, once an expandable segment of the planar frame, such as spiked cell 108a, is deployed out of the inner catheter 214, it can assume an expanded state. In some implementations, the planar frame 102 is a self-expandable frame which is shape-set to automatically expand as soon as it is uncovered by an outer cover, such as a capsule 218 or any other form of distal end portion 216 of inner catheter 214. Such a cover can be withdrawn proximally relative to the frame 102, or the frame 102 can be pushed out of the inner catheter 214. In some implementations, the planar frame 102 is made of a shape-memory material such as, but not limited to, nickel titanium alloy (e.g., Nitinol). When an expendable segment of the planar frame 102, such as expandable spiked cell 108, is deployed and is no longer constrained inside inner catheter 214, it can be free to assume a free state thereof, which can be, in some implementations, the expanded state described above with respect to FIG. 3B.

Deployment of the planar frame 102 can be performed in a gradual manner, such that partial deployment of one or more expandable segment(s), such as one or more spiked cell(s) 108, allows them to assume the free or expanded state, while the remainder expendable segments still residing inside the inner catheter lumen 220, such as spiked cell 108b shown in FIG. 4B. remain in a collapsed state, until further deployment gradually uncovers the entire length of the planar frame 102, such that all expandable segments (for example, all spiked cells 108) are in the expanded state.

FIGS. 5A-5D show at least some phases of an example method of using delivery assembly 200 to repair a native valve, such as mitral valve 36. In some implementations, delivery apparatus 202 can include an outer catheter 206 comprising a sheath configured for transluminal advancement through vasculature of a patient.

In some implementations, the outer catheter 206 can include a sheath configured for advancement through a femoral artery toward an interatrial portion of septum 48 of a heart 20 of a patient. A distal end portion 208 of the outer catheter 206 can be configured to pass through the interatrial septum of the subject, and to be oriented in a desired spatial orientation within the left atrium 32 (see FIG. 5A). In some implementations, the distal end portion 208 of outer catheter 206 is steerable. That is, the distal end portion 208 is deflectable with respect to an immediately more proximal portion of the outer catheter 206.

In some implementations, delivery apparatus 202 can further include a guide catheter 210 comprising a distal end portion 212 that is configured to pass through outer catheter 206 (i.e., through a primary lumen thereof), to become disposed outside of the distal end portion 208 of the outer catheter 206, and to be oriented in a desired spatial orientation within the left atrium 32 (see FIG. 5A). In some implementations, the distal end portion 212 of the guide catheter 210 is steerable. That is, the distal end portion 212 is deflectable with respect to an immediately more proximal portion of guide catheter 210. The guide catheter 210 can be steerable to a desired spatial orientation in order to facilitate advancing and implantation of an annuloplasty device 100.

In some implementations, the inner catheter 214, is configured to pass through the guide catheter 210 or the outer catheter 206 (i.e., through primary lumens thereof), such that its distal end portion 216 is configured to become disposed outside of the distal end portion 208 of the outer catheter 206 or the distal end portion 212 of the guide catheter 210, and to be oriented in a desired spatial orientation within the left atrium 32.

In some implementations, the distal end portion 216 of the inner catheter 214 is steerable. That is, the distal end portion 216 is deflectable with respect to an immediately more proximal portion of the inner catheter 214. The inner catheter 214 is steerable to a desired spatial orientation in order to facilitate advancing and implantation of an implant in a body cavity of the subject.

In some implementations, an implantation procedure begins by advancing a semi-rigid guidewire (not shown) into a right atrium 22 of a heart 20 of the patient. The procedure is typically performed with the aid of imaging, such as fluoroscopy, transesophageal echo, and/or echocardiography. The guidewire provides a guide for the subsequent advancement of the outer catheter 206 therealong and into the right atrium 22. In some implementations, once distal end portion 208 of catheter 206 has entered the right atrium 22, the guidewire is retracted from the subject's body.

For applications in which delivery apparatus 202 is used to deliver an annuloplasty device 100 to the mitral valve 36 of the patient, the outer catheter 206 is typically configured for initial advancement through the patient's vasculature into the right atrium 22 and through the septum 48, until the distal end portion 208 of the outer catheter 206 is positioned in the left atrium 32. The steerable distal end portion 208 of the outer catheter 206 is then steered such that it is positioned in a desired spatial orientation within the left atrium 32. The steering procedure is typically performed with the aid of imaging, such as fluoroscopy, transesophageal echo, and/or echocardiography.

In some implementations, the outer catheter 206 is advanced through the vasculature into the right atrium 22 using a suitable point of origin typically determined for a given patient. In some implementations, the outer catheter 206 is introduced into the femoral vein of the patient, through the inferior vena cava 12, into the right atrium 22, and into the left atrium 32 transseptally, typically through the fossa ovalis (hidden from view in FIG. 5A). In some implementations, the outer catheter 206 is introduced into the basilic vein, through the subclavian vein to the superior vena cava, into the right atrium 22, and into the left atrium 32 transseptally, typically through the fossa ovalis (not shown). In some implementations, the outer catheter 206 is introduced into the external jugular vein, through the subclavian vein to the superior vena cava, into the right atrium 22, and into the left atrium 32 transseptally, typically through the fossa ovalis (not shown). The outer catheter 206 is advanced distally until its distal end portion 208 reaches the interatrial septum, at which point the guidewire is withdrawn.

In some implementations, a resilient needle and a dilator (not shown) are advanced through the outer catheter 206 and into the heart 20. In order to advance the outer catheter 206 transseptally into the left atrium 32, the dilator is advanced to the septum 48, and the needle is pushed from within the dilator and is allowed to puncture the septum to create an opening that facilitates passage of the dilator and subsequently the outer catheter 206 therethrough and into the left atrium 32.

In some implementations, following the steering of the distal end portion 208 of the outer catheter 206, the guide catheter 210 is advanced through the outer catheter 206 in order to facilitate delivery and implantation of an annuloplasty device 100 along an annulus of the mitral valve 36, or to guide the inner catheter 214 thereto, as illustrated in FIG. 5A. In some implementations, the guide catheter 210 is a steerable catheter configured to guide the inner catheter 214 to the annulus 38. In some implementations, during the delivery, at least a portion of its steerable distal end portion 212 is exposed from the distal end portion 208 of the outer catheter 206 and is thus free for steering toward tissues of the heart 20, such as the annulus 38 of the mitral valve 36, as will be further described hereinbelow.

In some implementations, following the steering of the steerable distal end portion 212 of the guide catheter 210, the inner catheter 214 is advanced through the guide catheter 210 in order to facilitate delivery and implantation of annuloplasty device 100 along the annulus 38 of the mitral valve 36. In some implementations, during the delivery, at least a portion of the steerable distal end portion 216 of the inner catheter 214 is exposed from the distal end portion 212 of the guide catheter 210 and is thus free for steering toward a heart tissue, such as the annulus 38 of the mitral valve 36, as will be further described hereinbelow.

While methods of utilizing delivery assemblies 200 are described and illustrated herein with respect to implantation of annuloplasty devices 100 at a mitral valve 36, it is to be understood that any method for using delivery assemblies 200 and annuloplasty devices 100 can be similarly used for treatment of a tricuspid valve 26, wherein both valve 26, 36 can include leaflets that do not coapt properly and are in need of repair. For example, advancement of an inner catheter 214 towards the annulus of a tricuspid valve 26 can be performed in a similar manner to that described above with respect to the mitral valve 36, but without penetrating the septum, but rather, once positioned inside the right atrium 22, similarly steering any of the outer catheter 206, guide catheter 210, and/or inner catheter 214, to direct the distal end portion 216 towards the annulus of tricuspid valve 26.

In the enlarged view of FIG. 5A, the mitral valve 36 is shown to include native leaflets 40 surrounded by annulus 38, wherein leaflets 42 and 44 do not coapt properly, such that a gap exists in the valve 36 when it is closed. As further shown in FIG. 5A, the distal end portion 216 of inner catheter 214 is positioned upstream from the native valve. The term "native valve", as used herein, can refer to either the mitral valve 36 or tricuspid valve. The term "native leaflets", as used herein, can refer either to the anterior and posterior leaflets 42, 44 of the mitral valve, or to three native leaflets of a tricuspid valve, that can also form a gap therebetween when the valve is closed. Similarly, the term "annulus", as used herein, can refer either to an annulus of the mitral valve, surrounding the mitral leaflets, or an annulus of the tricuspid valve.

A part of the distal end portion 216 of inner catheter 214 is shown in FIG. 5A with partial transparency, to expose a portion of the planar frame 102 disposed therein. The distal end portion 216 can be positioned over the annulus 38 such that the plane defined by the distal opening 222 is orthogonal to, or angled relative to, the plane of the native annulus.

FIG. 5B shows a portion of planar frame 102^{a} deployed out of the inner catheter 214 and placed onto mitral valve 36, such as onto annulus 38. While the uncovered spiked cells 108 are shown in FIG. 5B to assume an expanded state as soon as they are deployed out of inner catheter 214, the spiked cells 108 that still remain inside inner catheter lumen 220 retain their collapsed state. The remaining spiked cells 108 are gradually uncovered, such as by retrieval of the inner catheter 214, until the entire planar frame 102^{a} is deployed and placed onto mitral valve 36, as shown in FIG. 5C. The tensioning member 138 is removed from view in FIGS. 5A and 5B for ease of illustration. While the term "placed onto", with reference to a native valve, may refer to placement onto an annulus of the native valve, in some implementations, this may also indicate placement onto a portion of one or more of the native leaflets, such as in the vicinity of the nadir of the leaflets.

The plane defined by the planar frame 102, or any spiked cell 108 thereof, is parallel to the plane of the annulus of the native valve. Thus, each spiked cell 108 is co-planarly placed onto mitral valve 36, such as onto its annulus 38, when deployed out of inner catheter 214. The spiked struts 104 comprise spikes 136 configured to penetrate into a tissue of a native valve and retain engagement therewith. For example, any spiked cell 108 placed onto mitral valve 36, engages the mitral valve 36 by corresponding spikes 136 directed towards, and optionally penetrating into, the tissue material of the native valve. In some cases, the expendable section of the deployed frame, such as a spiked cell 108, can first contact the mitral valve 36 while still in the collapsed state, and transition to the expanded state after being fully uncovered. Movement of spiked struts 104 towards each other during transitioning from a collapsed state to an expanded state, can pinch a portion of the tissue disposed between the struts, such as a portion of the tissue positioned inside the space defined by spiked cell(s) 108, causing the spikes 136 to press against, and optionally penetrate deeper into, the tissue, thereby increasing engagement with the tissue.

In some implementations, the spikes 136 extend from spiked struts 104 in a co-planar manner, such that the spikes 136 are co-planar with the plane defined by the planar frame 102 or any spiked cell 108 thereof, and/or co-planar with the plane of the annulus of the native leaflet. In some implementations, spikes 136 extend from one side of each spiked strut 104 but not from the other. For example, as shown in FIGS. 3A-4B, the spikes 136 in each spiked cell 108 can extend from sides of spiked struts 104 that face the inner space defined by the spiked cell 108, such that the spikes are directed towards each other, while the opposite sides of these spiked struts 104, facing the inner wall of the inner catheter 214, are devoid of spikes. This can protect the wall of the inner catheter 214 from being engaged by, and potentially scratched and damaged by, such spikes. Moreover, this arrangement of spikes 136 facing each other within each spiked cell 108 can facilitate engagement with a tissue contacted by the spikes, when the spiked cell 108 transitions from a collapsed state to an expanded state, and/or when the spiked cell 108 transitions from an expanded state to a contracted state. Nevertheless, in some implementations, struts can also include spikes extending from both sides.

In some implementations, once the spiked cells 108 of planar frame 102^{a} are placed over and are engaged with the mitral valve 36, the tensioning member 138 can be proximally pulled, tightening/contracting the tensioning member 138 in a manner that transitions the frame 102^{a} and spiked cells 108 thereof to the contracted state. This in turn decreases the size of the annulus 38, such that the native leaflets can properly coapt when the native annulus is closed, as illustrated in FIG. 5D.

In some implementations, delivery assembly 200 further comprises a locker 250, an example of which is schematically illustrated in FIGS. 5B-5D. Tensioning member 138 can extend through the locker 250, which can be proximal to the planar frame 102. In some implementations, locker 250 can be positioned distal to the deployment plug, such as distal to push member 228, as shown in FIG. 5B. In some implementations, transitioning of the frame 102 to the contracted state can be facilitated by distally pushing the locker 250 over tensioning member 138 against the planar frame 102, such as against the final junction 126 of final cell 128. This can be optionally accomplished by utilizing the push shaft 226 to distally push the locker 250. In some implementations, transitioning of the frame 102^{a} to the contracted state is facilitated by simultaneously pulling the tensioning member 138 and pushing the locker 250. The tensioning member can be then cut, for example at a free cut end 142 shown in FIG. 5D, with the locker 250 maintaining tension on the tensioning member 138 to retain the planar frame 102 in the contracted state, after which the delivery apparatus 202 can be retrieved from the patient's body. Examples of lockers that can be used to maintain tension of a tensioning member are presented in US application publication No. 2007/0276437, which is incorporated herein by reference.

In some implementations, the locker is implemented as a cutting and locking assembly 250, configured to lock the tensioning member 138 in position once the planar frame 102 assumes the contracted state. The cutting and locking assembly 250 can be configured to remain attached to the tensioning member 138 and retain it in a locked state, and also include a cutting or trimming mechanism, configured to cut the tensioning member 138 after being locked thereby. Further details regarding various configurations of lockers configured for locking and cutting tensioning members, are presented in full detail in US application Nos. 62/927,624 and 62/949,392, which is incorporated herein by reference.

While the planar frame 102^{a} is shown in FIGS. 5A-5D to extend along an arcuate path that spans less than 180°, optionally defining a central frame axis Ca that can lie beyond the anatomical boundaries of the native valve (see FIG. 5D), it is to be understood that this is shown by way of illustration and not limitation, and that a planar frame 102 can be released over any portion of the native valve, including any arcuate or curved path, and including circumscribing the native leaflets in a substantially circular manner (as illustrated in FIG. 2), elliptical manner, or any other curved path that can follow more than one radius of curvature at different sections thereof.

FIG. 6 shows an example planar frame 102^{b}, in an expanded state thereof. Planar frame 102^{b} is an example implementation of planar frame 102, and thus can include some or all of the features described for planar frame 102, except that planar frame 102^{b} includes a single rung 106 of spiked struts 104, that do not form closed cells. As shown, in such a case, the spiked struts 104 are arranges in a zig-zagged pattern, such that the junctions 112 include outer apices 114 and inner apices 116, without forming intermediate junctions.

In some implementations, planar frame 102^{b} includes at least two spiked struts 104, each of which is connected to an adjacent strut on one end, but remains unconnected to any other strut on the opposite intermediate junction. For example, planar frame 102^{b} is illustrated in FIG. 6 to include a first strut 124 attached to an adjacent strut by a junction 112 on one end thereof, while a junction 112, which can be also referred to as a first junction 120 of planar frame 102^{b}, remains free ended and unattached to any other spiked strut 104. Planar frame 102^{b} is also shown to include a final strut 130 attached to a preceding spiked strut 104 by a junction, but remaining free ended and unattached to any other strut at an opposite junction 112, which can be also referred to as a final junction 126 of planar frame 102^{b}.

In some implementations, a strut rung 106 can include a first strut 124 free ended at a first junction 120 and attached to a subsequent spiked strut 104 at a junction 112 opposite to the first junction 120, and a final strut 130 free ended at a final junction 126 and attached to a preceding spiked strut 104 at a junction 112 opposite to the final junction 126. Each of the spiked struts 104 disposed between the first strut 124 and the final strut 130 is attached to two adjacent spiked struts 104 at its opposite ends.

While the planar frame 102^{b} is illustrated in FIG. 6 to assume a substantially circular configuration, spanning almost 360° around the central frame axis Ca in the expanded and/or contracted state, it is to be understood that this is shown by way of illustration and not limitation, and that any other curved but not necessarily circular shape is contemplated, such as an arc shown in FIGS. 7A-7B for example.

As mentioned above, expandable segments of a planar frame 102 can independently transition, in some implementations, between collapsed and expanded states, irrespective of adjacent portions of the planar frame 102. For example, in a planar frame 102^{b} that includes a rung 106 of spiked strut 104, a coupled of two interconnected spiked struts 104 can transition between collapsed and expanded states thereof, while adjacent spiked struts 104 or couples of struts can assume or remain in a different state. In some implementations, the planar frame 102 and/or expandable segments thereof, such as any couple of spiked struts 104 of planar frame 102^{b}, can be configured to transition between a collapsed state, an expanded state, and a contracted state.

The term "expandable segment", as used herein, can refer to a portion or segment of the frame which is configured to transition between collapsed and expanded, and optionally contracted states as well. An expandable segment can be any spiked cell 108 of a planar frame 102^{a}, or any coupled of two interconnected spiked struts 104 of planar frame 102^{b}, for example.

A segmental length L in the case of a planar frame 102^{b}, can be defined as the distance between two subsequent inner apices 116 or the distance between two subsequent outer apices 114, which can be identical when all spiked struts 104 have the same lengths. A segmental width W in the case of a planar frame 102^{b} can be defined as the radial distance (along a radius extending from central frame axis Ca, for example) between an inner apex 116 and an outer apex 114 of the same spiked strut 104. In the collapsed state of an expandable segment of a planar frame 102^{b}, segmental length L1 can be greater than segmental width W1. In the expanded state, two subsequent inner apices 116 or two subsequent outer apices 114 of an expandable segment that includes two interconnected spiked struts 104 move closer to each other, such that segmental length L2 in the expanded state is less than segmental length L1 in the collapsed state, and segmental width W2 in the expanded state is greater than segmental width W1 in the collapsed state.

In some implementations, in the contracted state of planar frame 102^{b}, two subsequent inner apices 116 or two subsequent outer apices 114 of an expandable segment move further closer to each other, such that segmental length L3 in the contracted state is less than segmental length L2 in the expanded state, and segmental width W3 in the contracted state is greater than segmental width W2 in the expanded state.

In some implementations, annuloplasty device 100^{b} can further comprise tensioning member 138 attached at its attachment end portion 140 to an end of planar frame 102^{b}, such as to the first junction 120 (removed from view in FIG. 6, but shown in FIGS. 7A-7B). In some implementations, tensioning member 138 can extend along a series of subsequent outer apices 114 and/or inflow apices 116.

In some implementations, at least some of the outer apices 114 and/or inner apices 116 include guiding structures along which and/or through which the tensioning member 138 can extend. For example, at least some of the inner apices 116 of the example implementation of planar frame 102^{b} illustrated in FIG. 6 are shown to include eyelets 134 through which the tensioning member 138 can extend. In the illustrated example, all or most of the inner apices 116, excluding the first junction 120 and the final junction 126, can include such eyelets 134 through which the tensioning member 138 can proximally extend from the attachment end portion 140 at first junction 120. While inner apices 116 are shown in the illustrated example to include eyelets 134, it is to be understood that in some implementations, outer apices 114 include eyelets 134.

FIGS. 7A-7B show some phases of a method for implantation of an annuloplasty device 100^{b} that comprises planar frame 102^{b}. A delivery apparatus 202 can be utilized to deliver annuloplasty device 100^{b} towards a native valve, and deploy the planar frame 102^{b} out of inner catheter 214 to place it over the native valve, in the similar manner to that described above with respect to FIGS. 4A-5B, while FIGS. 7A and 7B illustrate phases of the method equivalent to those described above and illustrated in FIGS. 5C and 5D, respectively. For example, during gradual unsheathing of the planar frame 102^{b} to place it over a native valve such as mitral valve 36, any spiked strut 104 placed onto mitral valve engages the mitral valve 36 by corresponding spikes 136 directed towards, and optionally penetrating into, the tissue material of the native valve.

In some cases, the expendable section of the deployed frame, such as a couple of two interconnected spiked struts 104, can first contact the mitral valve 36 while still in the collapsed state, and transition to the expanded state after being during and/or following uncovering thereof. Pivotable movement of spiked struts 104 about their junctions 112 towards each other during transitioning from a collapsed state to an expanded state, can pinch a portion of the tissue disposed between the struts, such as a portion of the tissue positioned inside the space defined by spiked cell(s) 108, causing the spikes 136 to press against, and optionally penetrate deeper into, the tissue, thereby increasing engagement with the tissue. As mentioned above, spikes 136 can extend from one side of any spiked strut 104 of the plurality of spiked struts 104 as illustrated, or, in some implementations (not illustrated), from both sides of one or more of the plurality of spiked struts 104.

In some implementations, once the spiked struts 104 of planar frame 102^{b} are placed over and are engaged with the mitral valve 36, as shown in FIG. 7A, the tensioning member 138 can be proximally pulled, tightening/contracting the tensioning member 138 in a manner that transitions the frame 102^{b} and spiked struts 104 of its expandable segments to the contracted state. This in turn decreases the size of the annulus 38, such that the native leaflets can properly coapt when the native annulus is closed, as illustrated in FIG. 7B. Locker 250 can be utilized to retain the tensioning member 138 in a locked state, and to optionally cut or trim the slack of tensioning member 138 extending proximally from the locker 250, as described above.

While the planar frame 102^{b} is shown in FIGS. 7A-7B to extend along an arcuate path that spans less than 180°, optionally defining a central frame axis Ca that can lie beyond the anatomical boundaries of the native valve, it is to be understood that this is shown by way of illustration and not limitation, and that planar frame 102^{b} can be released over any portion of the native valve, including any arcuate or curved path, and including circumscribing the native leaflets in a substantially circular manner (as illustrated in FIG. 6), elliptical manner, or any other curved path that can follow more than one radius of curvature at different sections thereof.

While annuloplasty devices 100, including any of annuloplasty devices 100^{a} and 100^{b}, are described above to include planar frames 102 that can transition between collapsed states, expanded states, and constricted states, it is to be understood that in some implementations, annuloplasty device 100 can be devoid of a tensioning member 138, such that any of the planar frames 102, including planar frame 102^{a} or 102^{b}, can be configured to transition between collapsed and expanded states, but don't necessarily include any mechanism by which the frame can also transition to a constricted state.

For example, any expandable segment of a planar frame 102, such as any spiked cell 108 of planar frame 102^{a} or couple of interconnected spiked struts 104 of planar frame 102^{b}, can be delivered by the delivery apparatus 202 in a collapsed state toward the native valve, and deployed onto the native valve such that the spikes 136 contact and engage with the native tissue, and as soon as the expandable segment of the planar frame 102 transitions to the expanded state, the spikes 136 of adjacent spiked struts 104 move towards each other, such that the tissue of the native valve (such as of annulus 38) is drawn into the spaces between the adjacent spiked strut 104 (including the spaces defines by spiked cells 108), essentially plicating the tissue of the annulus 38 between spiked struts 104 that move towards each other when transitioning to the expanded state.

In some implementations, plication of the tissue of the native valve between adjacent spiked struts 104 moving towards each other when transitioning to an expanded state (i.e., when the segmental lengths L are shortened from L1 in the collapsed state to L2 in an expanded state), sufficiently contracts the annulus of the native valve in a manner to allows the native leaflets to properly coapt in the closed state of the valve, without the need for utilizing a tensioning member 138 or any other mechanism for further constriction of the frame 102.

While the planar frame 102 can be formed of a shape-memory material, pre-shaped to assume an expanded state during unsheathing from an inner catheter 214, it is to be understood that in some implementations, planar frame 102 can be formed of any other suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When formed of plastically deformable materials, the planar frame 102 and any expandable segments thereof are not necessarily shape-set to self-expand during unsheathing, by may be forced to transition to the expanded state (as well as to the constricted state) by other mechanisms, such as a tensioning member.

In some implementations, a pull force can be applied to tensioning member 138 while the planar frame 102 or any portion thereof, is still disposed inside the inner catheter 214. In such cases, while the planar frame 102, or any extendable section thereof, resides inside inner catheter 214, the internal boundaries of inner catheter 214 will keep the planar frame 102 or extendable section(s) thereof in a collapsed state, but any extendable section of the frame 102 uncovered and deployed out of the distal end portion 216 will be forced to expand due to the proximally-oriented tensioning of the tensioning member 138.

In some implementations, pull force can be applied to tensioning member 138 during or after deployment of any extendable section of the frame 102, or following full deployment of the entire frame 102. For example, planar frame 102 or any expandable segment thereof can be deployed and placed on the native valve while still in the collapsed state, after which the tensioning member 138 can be proximally pulled to expand it. When tensioning member 138 is used to transition the frame 102 or any extendable portion thereof, from the collapsed state, it can transition it directly to another state which can be referred to as an expanded state, but may have segmental length and width equivalent to those described above for a constricted state.

FIGS. 8A and 8B show a side view and a top view, respectively, of an example annuloplasty device 100^{c}. In some implementations, annuloplasty device 100^{c} comprises a planar frame 102^{c} coupled to a cylindrical frame 150. Planar frame 102^{c} is an example implementation of planar frame 102, and thus can include some or all of the features described for planar frame 102, except that planar frame 102^{c} is attached, along inner apices 116, to cylindrical frame 150, and can be formed of a continuous row 110 of spiked cells 108 or a continuous rung 106 of spiked struts 104 that do not include free ends, such as in the case of example planar frames 102^{a} and 102^{b} illustrated to be free ended at their first junction 120 and final junction 126 in FIG. 2 and FIG. 6, respectively.

It is understood that annuloplasty devices disclosed herein that include a cylindrical frame 150, may be used with a variety of implant delivery apparatuses. In some implementations, a cylindrical frame is a balloon expandable cylindrical frame, which generally involves a procedure of inflating a balloon within the cylindrical frame, thereby expanding the cylindrical frame at the desired site of implantation. In some implementations, once the cylindrical frame is sufficiently expanded, the balloon is deflated and retrieved along with the delivery apparatus.

In some implementations, a cylindrical frame is a self-expandable, which includes a frame that is shape-set to automatically expand as soon an outer retaining shaft or capsule (such as a capsule 218 or distal end portion 216 of an inner catheter 214) is withdrawn proximally relative to the cylindrical frame.

In some implementations, a cylindrical frame is a mechanically expandable cylindrical frame, which generally relies on a mechanical actuation mechanism for expansion. The mechanical actuation mechanism can include a plurality of expansion and locking assemblies (such as the mechanical expansion mechanisms described in U.S. Patent No. 10,603,165, International Application No. PCT/US2021/052745 and U.S. Provisional Application Nos. 63/85,947 and 63/209904, each of which is incorporated herein by reference in its entirety), releasably coupled to respective actuation assemblies of a delivery apparatus, controlled via a handle (not shown) for actuating the expansion and locking assemblies to expand the cylindrical frame to a desired diameter. In some implementations, the expansion and locking assemblies may optionally lock the cylindrical frame's diameter to prevent undesired recompression thereof, and disconnection of the actuation assemblies from the expansion and locking assemblies, to enable retrieval of the delivery apparatus once the annuloplasty device is properly positioned at the desired site of implantation.

The cylindrical frame 150 can comprise an outflow end 154 and an inflow end 152. In some instances, the outflow end 154 is the distal end of the cylindrical frame 150, and the inflow end 152 is the proximal end of the cylindrical frame 150. Alternatively, depending for example on the delivery approach of the annuloplasty device, the outflow end can be the proximal end of the outflow end, and the inflow end can be the distal end of the outflow end.

The term "outflow", as used herein, refers to a region of the cylindrical frame through which the blood flows through and out of the annuloplasty device 100.

The term "inflow", as used herein, refers to a region of the cylindrical frame through which the blood flows into the annuloplasty device 100.

In some implementations, the cylindrical frame 150 is movable between a radially compressed configuration and a radially expanded configuration. The cylindrical frame 150 can be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a plastically-deformable materials, the cylindrical frame 150 can be crimped to a radially compressed configuration on a balloon catheter 236 (shown, for example, in FIG. 15B), and then expanded inside a patient by an inflatable balloon 238 (shown, for example, in FIGS. 15A-15B) or equivalent expansion mechanism. Alternatively or additionally, the cylindrical frame 150 can be made of shape-memory materials such as, but not limited to, nickel titanium alloy (e.g., Nitinol). When constructed of a shape-memory material, the cylindrical frame 150 can be crimped to a radially compressed configuration and restrained in the compressed configuration by insertion into a shaft (such as inner catheter 214) or equivalent mechanism of a delivery apparatus.

In the example illustrated in FIGS. 8A-8B, the cylindrical frame 150 is an annular, stent-like structure comprising a plurality of intersecting angled struts 156. The cylindrical frame 150 can include a plurality of strut rungs that can collectively define one or more rows of cells 158. The cylindrical frame 150 can have a cylindrical or substantially cylindrical shape having a constant diameter from the inflow end 152 to the outflow end 154 as shown, or the frame can vary in diameter along the height of the cylindrical frame, as disclosed in US Pat. No. 9,155,619, which is incorporated herein by reference. In some implementations, the cylindrical frame 150 includes a single row of cells 158, as illustrated. In some implementations, the cylindrical frame 150 can include more than one row of cells 158.

In some implementations, at least some of the angled struts 156 can be pivotable or bendable relative to each other, so as to permit expansion or compression of cylindrical frame 150. For example, the cylindrical frame 150 can be formed from a single piece of material, such as a metal tube, via various processes such as, but not limited to, laser cutting, electroforming, and/or physical vapor deposition, while retaining the ability to collapse/expand radially in the absence of hinges and the like.

In some implementations, the end portions of the angled struts 156 intersect at inflow junctions 160 at the inflow end 152 and at outflow junctions 162 at the outflow end 154. In some implementations, the angled struts 156 can intersect at additional junctions formed between the inflow junctions 160 and the outflow junctions 162. In some implementations, the cylindrical frame 150 comprises a single row of cells 158, as illustrated. In some implementations, the cylindrical frame can include more than one row of cells.

As mentioned above, the planar frame 102 of annuloplasty device 100^{c} is coupled to the cylindrical frame 150, optionally to the inflow end 152 or to the outflow end 154. In some implementations, planar frame 102 is coupled to the outflow end 154 of cylindrical frame 150. In some implementations, the inner apices 116 of planar frame 102 are coupled to the outflow junctions 162 of cylindrical frame 150. In some implementations, the planar frame 102 is integrally formed with the cylindrical frame 150, such that the inner apices 116 of the planar frame 102 coincide with or are integrally formed with the outflow junctions 162 of cylindrical frame 150. In some implementations, the planar frame 102 and the cylindrical frame 150 are provided as to separate components which are attached to each other, for example by welding, gluing, suturing or attaching by any other means known in the art, the inner apices 116 to the outflow junctions 162.

In some implementations, the central frame axis Ca of planar frame 102 is also the central longitudinal axis of the cylindrical frame 150, extending from the inflow end 152 to the outflow end 154, as illustrated. When the frames 102, 150 are attached to each other, the planar frame 102 is substantially orthogonal to the cylindrical frame 150 and central axis Ca. For example, a planar frame 102 can be perpendicular to cylindrical frame 150 and central axis Ca, or deviate from a plane that is perfectly perpendicular to cylindrical frame 150 and central axis Ca by up to 5°, 10°, 15°, or 20°.

FIG. 8A illustrates an example cylindrical frame 150^{c}, which is an example implementation of cylindrical frame 150 and thus can include some or all of the features described for cylindrical frame 150, except that the inflow junctions 160^{c} of cylindrical frame 150^{c} are free ended, with no additional struts or constructs extending therefrom.

In some implementations, planar frame 102^{c} further comprises a tensioning member 138, as shown in FIG. 9. In some implementations, a planar frame 102^{c} can include a row 110 of spiked cells 108 such that each of the spiked cells 108 is connected on both sides thereof, to adjacent spikes cells 108. That is to say, all intermediate junction 118 can be disposed between two spikes cells 108, without including a free ended first junction or a free ended final junction. In such cases, the tensioning member 138 can be connected, at its attachment end portion 140, to one of the intermediate junctions 118, as illustrated. The tensioning member 138 can extend along or be otherwise coupled to all other intermediate junctions 118, including by passing through channels 132 or any other suitable manner.

In some implementations, the attachment end portion 140 comprises a loop around an intermediate junction, such that the tensioning member 138 can extend along the circumference of the planar frame 102^{c}, pass through the same loop formed by the attachment end portion 140, and extend proximally therefrom, such as towards a handle (not shown) of the delivery apparatus. In some implementations, the tensioning member 138 can extend through a locker 250 that can be implemented according to any of the examples described above.

While a planar frame 102^{c} is shown to include a rung 106 of spiked cells 108, similar to the spiked cells 108 described above with respect to planar frame 102^{a}, it is to be understood that in some implementations, a planar frame 102^{c} can include a single rung 106 of spiked struts 104, similar to the rung 106 of spiked struts 104 described above with respect to planar frame 102^{b}. In some implementations, planar frame 102^{c} can include a rung 106 of spiked struts 104 such that each of the spikes struts 104 is connected on both sides thereof, to adjacent spikes struts 104. That is to say, at least two spiked struts 104 diverge from each of the outer apices 114 and from each of the inner apices 116, without including a free ended first junction or a free ended final junction. In such cases, a tensioning member 138 can be connected, at its attachment end portion 140, to one of the outer apices 114 or to one of the inner apices 116. The tensioning member 138 can extend along or be otherwise coupled to all other outer apices 114 and/or inner apices 116, including by passing through eyelets 134 or in any other suitable manner.

In some implementations, the attachment end portion 140 comprises a loop or knot attached to a junction 112, which can be either an outer apex 114 or an inner apex 116, such that tensioning member 138 can extend therefrom along the circumference of the planar frame, pass back through the same eyelet 134 to which the attachment end portion 140 is coupled, and extend proximally therefrom.

FIGS. 10A-10B show cross-sectional views of a distal portion of an example delivery assembly 200^{c} that includes an example delivery apparatus 202^{c} adapted to deliver and implant annuloplasty device 100^{c}. Delivery apparatus 202^{c} is an example implementation of delivery apparatus 202, and thus can include some or all of the features described for delivery apparatus 202, except that the inner catheter lumen 220 of inner catheter 214^{c} can be sized to accommodate annuloplasty device 100^{c} therein, in a radially compressed configuration of the cylindrical frame 150, and the deployment plug 228^{c} at the end of deployment shaft 226^{c} can be similarly sized to contact and support and/or push against the inflow end 152 of cylindrical frame 150.

FIG. 10A shows the annuloplasty device 100^{c} retained entirely inside inner catheter 214^{c}, optionally within capsule 218 or otherwise structured distal end portion 216 of inner catheter 214^{c}. The cylindrical frame 150 is kept in the radially compressed configuration during delivery inside the inner catheter lumen 220, and is proximal to the planar frame 102, which is also retained in a collapsed state during delivery while residing inside the inner catheter 214^{c}.

In some implementations, a planar frame 102 coupled to a cylindrical frame 150, such as the planar frame 102^{c}, is configured to transition between an axially aligned state and a planar state. In the aligned state, the outer apices 114 of the planar frame 102^{c} are axially aligned with the outflow junctions 162 of the cylindrical frame 150. In some implementations, in the axially aligned state, the outer apices 114 and inner apices 116 are axially aligned with the inflow junctions 160 and outflow junction 162. In the axially aligned state, an imaginary line extending from each outflow junction 162 to a corresponding outer apex 114, and/or through the corresponding inflow junction 160 and/or inner apex 116, is parallel to the central longitudinal axis Ca. In the planar state, the outer apices 114 of the planar frame 102^{c} are positioned radially away from the outflow junctions 162 of the cylindrical frame 150, optionally while being at the same axial level as that of the outflow junctions 162 relative to the central longitudinal axis Ca. This means that an imaginary line passing between any inner apex 116 and outer apex 114 of an expendable segment of the planar frame 102^{c}, is substantially perpendicular to (or slightly angled relative to, for example by up to 5°, 10°, 15°or 20°) an imaginary line passing between the inflow junction 160 and outflow junction 162 of a cell 158 attached to the corresponding expandable segment.

The planar frame 102^{c} can be formed of a shape-memory material (such as Nitinol), and shape-set to extend radially outward from the cylindrical frame 150, along a plane that is substantially orthogonal to a central longitudinal axis Ca of the cylindrical frame 150, configured to assume a planar state when not bound by an external restricting component. As shown in FIG. 10A, while both the cylindrical frame 150 and planar frame 102^{c} reside inside the inner catheter 214^{c}, the cylindrical frame 150 is in a radially compressed configuration and the planar frame 102^{c} is in an axially aligned state.

Since the outflow junctions 162 are connected to the inner apices 116, the circumferential distance between adjacent outflow junctions 162 is also the circumferential distance between adjacent inner apices 116, which similarly corresponds to the circumferential distance between adjacent intermediate junction 118. Thus, the configuration or diameter of the cylindrical frame 150 dictates the segmental lengths L between adjacent intermediate junctions 118, as well as the distances between adjacent inner apices 116 and the distances between adjacent outer apices 114.

When the planar frame 102^{c} is in the axially aligned state, it is also in a collapsed state. As shown in FIG. 10A, when the cylindrical frame 150 is in the crimped or radially compressed configuration, adjacent outflow junctions 162 thereof are kept relatively closer to each other than in a radially expanded configuration, which will cause adjacent intermediate junctions 118 of the planar frame 102^{c} to be also kept closer to each other, forming a segmental length L0 in the collapsed state of the planar frame 102^{c}.

As shown in FIG. 10B, the annuloplasty device 100^{c} can be partially expelled out of inner catheter 214^{c}, for example by utilizing the push member 228^{c} of a push shaft 226^{c} that prevents proximally-oriented movement of the annuloplasty device 100^{c} while the inner catheter 214^{c} is proximally pulled to uncover planar frame 102^{c}, and/or distally moving the push member 228^{c} of push shaft 226^{c} to urge the planar frame 102^{c} out of the inner catheter 214^{c}. As the planar frame 102^{c} is freed from inner catheter 214^{c}, it springs radially outward to assume its pre-shaped planar state, orthogonal to the cylindrical frame 150.

In some implementations, while the annuloplasty device 100^{c} is only partially expelled out of inner catheter 214^{c}, such that the planar frame 102^{c} is freed from the inner catheter 214^{c} and at least a portion of cylindrical frame 150 is still restricted to the radially compressed configuration inside the inner catheter 214^{c}, the segmental lengths L0 between adjacent intermediate junctions 118 remain unchanged, since the diameter of the cylindrical frame 150 has not been changed. Thus, while the planar frame 102 transitions from an axially aligned state in FIG. 10A to a planar state in FIG. 10B, as long as the cylindrical frame 150 attached thereto remains in a radially compressed configuration, the planar frame 102 remains in the compacted state.

Further deployment of the annuloplasty device 100^{c} out of inner catheter 214^{c}, such as by pushing the push shaft 226^{c} and/or pulling the inner catheter 214^{c} to uncover the cylindrical frame 150, will allow expansion of the cylindrical frame 150 by any suitable mechanism, such as by allowing it to self-expand if formed of a shape-memory material, or by employing other expanding mechanisms such as balloon inflation or mechanical expansion, as described above.

In some implementations, when cylindrical frame 150 is expanded, adjacent outflow junctions 162 move away from each other, transitioning the planar frame 102^{c} therewith to the expanded state, similarly enlarging the segmental lengths to L2 between adjacent intermediate junctions 118, in a manner similar to that illustrated in FIGS. 8B and 9. As evident from FIGS. 10A and 10B, in the case of a planar frame 102 attached to cylindrical frame 150, such as planar frame 102^{c}, the segmental length L0 in the compacted state is less than the segmental length L2 in the expanded state.

Thus, a planar frame 102^{c} can be in any of the following combination of states: (a) a collapsed state while in an axially aligned state, as shown in FIG. 10A when the cylindrical frame 150 is in a radially compressed configuration, and the entire annuloplasty device 100 is retained inside the inner catheter 214; (b) a collapsed state while in the planar state, as shown in FIG. 10B when the cylindrical frame 150 is in a radially compressed configuration, and the planar frame 102^{c} is freed from the inner catheter 214, positioned distal to distal opening 222; and (c) an expanded state while in the planar state, as shown in FIGS. 8B and 9, such as when the entire annuloplasty device 100 is freed from the inner catheter 214 and the cylindrical frame 150 is in a radially expanded configuration. In some implementations, the planar frame 102^{c} can also assume (d) a constricted state while in the planar state, for example by, subsequent of assuming the state (c) mentioned above, a tensioning member 138 coupled to the planar frame 102^{c} (see FIG. 9) is tensioned, as will be further described below.

FIGS. 11A-11B show some phases of an example method of using delivery assembly 200^{c} to implant an annuloplasty device 100 that comprises planar frame 102 coupled to a cylindrical frame 150, such as annuloplasty device 100^{c}. In some implementations, delivery apparatus 202^{c} can be utilized to deliver a annuloplasty device 100, such as annuloplasty device 100^{c}, towards a native valve (such as the mitral valve 36 in the example illustrated in FIGS. 11A-11B), in the similar manner to that described above with respect to FIG. 5A, with the exception that the distal end portion 216 of the inner catheter 214 is not positioned over the annulus 38 such that the plane defined by the distal opening 222 is substantially orthogonal to the plane of the native annulus, but rather positioned above the mitral valve 36 such that the plane defined by the distal opening 222 is substantially parallel to (or slightly angled relative to) the plane of the native annulus.

In some implementations, a part of the distal end portion 216 of inner catheter 214 is shown in FIG. 11A with partial transparency, to expose components of the annuloplasty device 100 and delivery apparatus 202 disposed therein.

The term "above", as used herein with respect to components of the delivery assembly 200 positioned above the mitral valve 36, refers to a position in the atrial side of the valve, i.e. – within the left atrium 32. When delivery assembly 200 is utilized to repair the tricuspid valve 26, the term "above" will refer to a position within the right atrium 22.

In some implementations, when the distal opening 222 is positioned above the mitral valve 36, delivery apparatus 202^{c} can be utilized to deploy at least a portion of the annuloplasty device 100^{.} For example, as shown in FIG. 11A, annuloplasty device 100^{c} can be partially expelled out of inner catheter 214^{c}, such that the planar frame 102 is unsheathed and assumes a planar state, extending radially away from the cylindrical frame 150, while the cylindrical frame 150 can still remain in a radially compressed configuration and keeping the planar frame 102 in the collapsed state, as described above with respect to FIG. 10B. In some implementations, the distal end portion 216 of inner catheter 214 can be steered in this state to position the planar frame 102 at the level of the annulus 38, such that the spikes 136 can contact and engage with the tissue of the annulus 38, after which the cylindrical frame 150 can be expelled as well (for example, by retracting the inner catheter 214), allowed to expand and transition the planar frame 102 to its expanded state therewith.

In some implementations, the whole of the annuloplasty device 100, including cylindrical frame 150, is deployed out of the inner catheter 214 without partially deploying only the planar frame 102 and steering the inner catheter 214 prior to subsequent deployment of cylindrical frame 150. In some implementations, deployment of annuloplasty device 100^{c} can be performed at a position of the inner catheter 214 that facilitates contact of the planar frame 102 with the annulus 38 upon uncovering and/or expansion thereof.

In some implementations, a balloon expandable annuloplasty device 100 can be deployed out of the inner catheter 214 above the mitral valve 36, wherein an inflatable balloon 238 (illustrated, for example, in FIGS. 15A-15B) can be inflated inside the cylindrical frame 150, expanding the frame 150 while keeping it disposed therearound, allowing the annuloplasty device 100^{c} to be navigated towards the mitral valve 36 such that the planar frame 102 contacts and engages the annulus 38, for example by steering the balloon catheter 236 (illustrated, for example, in FIGS. 15A-15B) of a catheter disposed therearound, after which the balloon 238 can be deflated to decouple the annuloplasty device 100^{c} therefrom.

In some implementations, a mechanically expandable annuloplasty device 100 can be deployed out of the inner catheter 214 above the mitral valve 36, wherein actuators of the cylindrical frame (not shown) can be coupled to driver arms (not shown) of the delivery apparatus, utilized to expand the cylindrical frame 150. The annuloplasty device 100 can remain coupled, via such driver arms, to the delivery apparatus, which can be utilized to navigate the annuloplasty device 100^{c} towards the mitral valve 36 such that the planar frame 102 contacts and engages the annulus 38, after which such driver arms can be decoupled from the annuloplasty device 100^{c}.

Since the cylindrical frame 150 mainly serves to facilitate expansion of the planar frame 102, its height, defined between the inflow end 152 and the outflow end 154 can be kept to a minimum. In some implementations, the cylindrical frame 150 includes single row of cells 158. In some implementations, the distance between the inflow junctions 160 and outflow junctions 162 of cells 158, in an expanded configuration of the cylindrical frame 150, is less than the segmental width W of the planar frame 102, in an expanded and/or constricted state thereof.

While not illustrated in FIGS. 11A-11B, it is to be understood that an annuloplasty device 100^{c} can include a tensioning member 138, as described above with respect to FIG. 9. In such examples, the tensioning member 138 can be proximally pulled, tightening/contracting the tensioning member 138 in a manner that transitions the frame 102^{c} and spiked cells 108 and/or spiked struts 104 of its expandable segments to a contracted state. This in turn decreases the size of the annulus 38, such that the native leaflets can properly coapt when the native annulus is closed (see FIG. 11B). A locker 250 (not shown in FIGS. 11A-11B, but similar to the locker of FIGS. 5A-5D and 7A-7B) can be utilized to retain the tensioning member 138 in a locked state, and to optionally cut or trim the slack of tensioning member 138 extending proximally from the locker 250, as described above.

FIG. 11B shows the annuloplasty device 100 (such as annuloplasty device 100^{c}) implanted such that the native leaflets can properly coapt when the valve is closed, with the delivery apparatus 202 retrieved from the patient's body after implantation. In some implementations, as illustrated, the annuloplasty device 100 is implanted such that the planar frame 102 is attached to the atrial side of the native valve, and the cylindrical frame 150 extends from the planar frame 102 into the atrium (such as the left atrium 32 in the case of implantation at a mitral valve 36). Since the native leaflets 40 are oriented towards the ventricle 34, this position of the cylindrical frame 150 will prevent it from interfering with the movement of the leaflets between the open and closed states of the valve.

FIG. 12 shows a side view of an annuloplasty device 100^{d}, which is similar to any example described above with respect to annuloplasty device 100^{c}, including a planar frame 102 attached to a cylindrical frame 150, except that the cylindrical frame 150^{d} of annuloplasty device 100^{d} further comprises a plurality of engaging struts 164 extending proximally from the inflow junctions 160. Each engaging strut 164 can terminate at a proximal end thereof with a tab 166, which can be mushroom-shaped as illustrated, or can have any other shape that is laterally wider than the remainder of the engaging strut 164.

FIG. 13 shows a cross-sectional view of a distal portion of a delivery assembly 200^{d}, illustrating the annuloplasty device 100^{d} of FIG. 12 retained, in a radially compressed configuration of the cylindrical frame 150^{d}, inside the inner catheter 214^{d} of delivery apparatus 202^{d}. Delivery apparatus 202^{d} can be similar to any example described above for delivery apparatus 202^{c}, except that the deployment plug 228^{d} can further include a plurality of slots 230 located between radially extending teeth 232 therebetween. In some implementations, the slots 230 are configured to receive and engage the engaging struts 164, and the tabs 166 can be retained within a circumferential gap 234 or ring located proximal to the radially extending teeth 232. While not illustrated in FIG. 13, in some implementations, the delivery apparatus 202^{d} can further comprises an outer retention member, which can be in the form of a cylinder, ring, and the like, extending around and distally from the deployment plug 228^{d}, to cover at least a proximal portion of the cylindrical frame 150^{d} while secured to the deployment plug 228^{d}.

In some implementations, while annuloplasty device 100^{d} remains coupled to deployment plug 228^{d}, the deployment shaft 226^{d} can be optionally maneuvered to properly position the annuloplasty device 100^{d}, such as by placing the planar frame 102 over and in contact with the annulus 38. For example, if further equipped with an outer retention member disposed around a proximal portion of the cylindrical frame 150^{d}, the annuloplasty device 100^{d} can be completely disposed out of the inner catheter 214^{d} while the cylindrical frame 150^{d} remains in a radially compressed configuration inside the outer retention member, and is coupled to the deployment plug 228^{d} by the engaging struts 164. In some implementations, after proper placement, the cylindrical frame 150^{d} can be uncovered, such as by retraction of the outer retention member, allowing the engaging struts 164 to spring out of the slots 230, thus decoupling the cylindrical frame 150^{d} from the deployment plug 228^{d} as it expands.

While engaging struts 164 are described herein and illustrated with respect to a cylindrical frame 150, it is to be understood that in some implementations, a similarly formed engaging strut, that can include a tab, can extend from a final junction 126 of a planar frame that can be similar to any of the example planar frame 102^{a} or 102^{b} described above. In such cases, the planar frame is disposed inside an inner catheter 214 of a delivery apparatus 202, that can be generally similar to delivery apparatus 202^{a} described above, during delivery towards the native annulus, the engaging strut can extend proximally from the final junction of the planar frame, and engage with a deployment plug 228 that can include similarly formed slots 230 that define radially extending teeth 232. This will allow for optional retraction of the annular frame 102 back into the inner catheter 214, after full or partial deployment thereof.

Any delivery apparatus disclosed herein can further include a nosecone 240 coupled to a nosecone shaft 242 that extends therefrom towards the handle (not shown). Wherein the nosecone 240, which can have a generally tapered distal end, can be positioned distal to the annuloplasty device 100 and any catheter of the delivery apparatus during delivery, configured to facilitate advancement of the delivery assembly 200 through the patient's vasculature. While illustrated only in FIG. 13, it is to be understood that any other example of delivery apparatus 202 disclosed throughout the current specification can include the nosecone 240 and the nosecone shaft 242. The nosecone shaft 242 can be axially movable relative to any other catheter or shaft of delivery apparatus 202, such as outer catheter 206, guide catheter 210, inner catheter 214, deployment shaft 226, and the like. For example, a deployment plug 228 can include a central channel through which the nosecone shaft 242 can extend, and the deployment shaft 226 can include a lumen through which the nosecone shaft 242 extends.

In some implementations, the inner apices 116 of planar frame 102 are affixed to outflow junctions 162 of cylindrical frame 150, such as by welding, gluing, and the like. In some implementations, the inner apices 116 of planar frame 102 and the outflow junctions 162 of cylindrical frame 150 are integrally formed, such as when both the planar frame 102 and the cylindrical frame 150 are integrally formed (for example, laser cut from the same piece of material). In some implementations, the inner apices 116 of planar frame 102 are also the outflow junctions 162 of cylindrical frame 150. It is to be understood that regardless of whether the inner apices 116 are attached to or integrally formed with the outflow junctions 162, attachment of the planar frame 102 to the cylindrical frame 150 is configured to allow pivotable movement of the planar frame 102 relative to the cylindrical frame 150 at the points of attachment, including any of the inner apices 116 and/or outflow junctions 162, to allow angular movement of the planar frame 102 from alignment with the cylindrical frame 150, as shown for example in FIGS. 10A or 13, to a planar configuration extending radially away from the cylindrical frame 150, as shown for example in FIG. 10B.

FIGS. 14A and 14B show a side view and a top view, respectively, of an example annuloplasty device 100^{e}, comprising. Annuloplasty device 100^{e} is to any annuloplasty device 100 that comprises a planar frame 102 coupled to a cylindrical frame 150, including any example described above with respect to annuloplasty devices 100^{c} and 100^{d}, except that annuloplasty device 100^{e} further comprises a plurality of connectors 170 coupling the planar frame 102 to the cylindrical frame 150, while allowing for pivotable movement therebetween. In some implementations, the connectors 170 comprise suture loops, looped around the inner apices 116 of planar frame 102 and the corresponding outflow junctions 162 of cylindrical frame 150, as illustrated.

When the planar frame 102 and cylindrical frame 150 are provided as separate components, coupled to each other by connectors 170, they can be formed from different materials. In some implementations, the cylindrical frame 150 is made of a shape-memory material configured to self-expand, while the planar frame 102 can be formed from any other type of material, including plastically deformable materials. In such examples, when the cylindrical frame 150 is unsheathed and allowed to self-expand, it will facilitate transitioning of the planar frame 102 to the expanded state as well.

In some implementations, the planar frame 102 is made of a shape-memory material configured to self-expand, while the cylindrical frame 150 can be formed from any other type of material, including plastically deformable materials. In such examples, the planar frame will strive to transition to the expanded state as soon as it's released from inner catheter 214 and allowed to assume its planar configuration, while the cylindrical frame 150 will prevent the planar frame 102 from expanding as long as the cylindrical frame 150 is radially compressed inside the inner catheter 214 or any other retaining member. However, as soon as the cylindrical frame 150 is uncovered, the planar frame 102 will be free to transition to its expanded state, forcing the cylindrical frame 150 to radially expand therewith.

FIGS. 15A-15B show some phases of an example method of using delivery assembly 200^{e} to implant an annuloplasty device 100, such as annuloplasty device 100^{e}. Delivery apparatus 202^{e} of delivery assembly 200^{e} can be similar to any other example of a delivery apparatus disclosed herein, except that delivery assembly 200^{e} further comprises a balloon catheter 236 having an inflatable balloon 238 mounted on its distal end. A balloon expandable cylindrical frame 150 of annuloplasty device 100 can be carried in a crimped or radially compressed configuration, over the balloon catheter 236 (including around inflatable balloon 238).

As shown in FIG. 15A, when reaching the site of implantation (for example, mitral valve 36), the deflated balloon 238, carrying crimped cylindrical frame 150 of annuloplasty device 100 thereover, can be advanced in some implementations, to position the planar frame 102 at or in close proximity to the annulus 38, after which the balloon can be inflated to radially expand the cylindrical frame 150, as shown in FIG. 15B, while the planar frame 102 can engage the annulus 38, or improve engagement if it is already in contact with the tissue of the annulus 38. The balloon 238 can be them deflated, and the delivery apparatus 202^{e} can be retrieved from the patient's body, optionally after further constricting the annulus 38 and planar frame 102 by tensioning and locking a tensioning member 138 (not shown in FIGS. 15A-15B).

In some implementations, the annuloplasty device 100 can be placed over the native valve, such that the planar frame 102 is initially engaged with the annulus, prior to balloon inflation. In some implementations, the annuloplasty device 100 can be at least partially expanded prior to balloon inflation, with the planar frame 102 engaged with the annulus, optionally by having at least one of the planar frame 102 or cylindrical frame 150 formed from a self-expanding shape-memory material. In such examples, a balloon 238, positioned in an inflated state inside the cylindrical frame 150, can be inflated so as to further expand the cylindrical frame 150 to a greater diameter.

FIG. 16A shows a bottom view of an example annuloplasty device 100^{f}. FIG. 16B shows a cross-sectional view taken along line FIGS. 16B-16B of FIG. 16A. Annuloplasty device 100^{f} is an example implementation of annuloplasty device 100, and thus can include some or all of the features described for annuloplasty device 100, except that annuloplasty device 100^{f} further includes a strip 180 coupled to the annular frame 102. In some implementations, the strip 180 is attached to one planar side of the planar frame 102, such that when the planar frame 102 is in an expanded state, as shown in FIG. 16A, the strip 180 also extends along the plane defined by the planar frame 102. In some implementations, the strip 180 can include a plurality of yarns or fibers that extend toward the annulus 38 once the annuloplasty device 100^{f} is implanted, configured to increase the surface area available for thrombogenesis and tissue growth.

Strip 180 can include a fabric body formed from a plurality of strands or yarns that are woven, knitted, or otherwise secured together. In some implementations, the strip 180 can comprise a woven or knitted fabric comprising a fabric base layer 182 and a plurality of pile yarns (e.g., loop yarn) or floating yarns 184 that extend away from the frame base layer 182. For example, the base layer 182 can comprise warp and weft yarns knitted into a mesh-like structure, and the pile yarns or floating yarns 184 can be knit or woven into and extend outward from the base layer 182. Further details on strips or fabrics that comprise a knitted or woven material and pile or floating yarns are disclosed in U.S. Patent Publication 2019/0374337 and International Patent Publication WO 2021/202636, which are incorporated by reference herein.

As illustrated, the fabric base layer 182 is disposed over, and coupled to, a surface of the planar frame 102 which is opposite to the surface configured to press against the annulus once the annuloplasty device 100^{f} is implanted. For example, when a planar frame 102 of annuloplasty device 100^{f}is engaged with a mitral valve 36, the fabric base layer 182 is disposed over the surface of the planar frame 102 facing the left atrium 32. In some implementations, the floating yarns 184 extend from the fabric base layer 182, through the spiked cells 108 for example, towards the tissue of the annulus. This configuration can promote tissue ingrowth around the planar frame 102, which can advantageously result in improved engagement, over time, of the planar frame 102 with the native annulus.

The yarns 184 can comprise any of various biocompatible thermoplastic polymers such as PET, Nylon, ePTFE, UHMWPE, etc., or other suitable natural or synthetic fibers. In some implementations, the yarns 184 can be woven on a loom, and can then be heat-treated or heat-set to achieve the desired size and configuration. For example, depending upon the material selected, heat-setting can cause a texturizing effect, or increase the amount of texturizing, of the yarns 184. Heat setting can also induce thrombogenic characteristics to the polymer surface, which may be beneficial for attachment to the annular tissue.

FIG. 16A illustrates an example annuloplasty device 100^{f} that includes a strip 180 coupled to a planar frame 102^{f} that can be similar to planar frame 102^{a} described above with respect to FIG. 2, in which case the strip can be an elongated rectangular or otherwise-shaped elongated strip that has two ends, attached to a first cell 128 and a final cell 128, that can be spaced from each other. However, it is to be understood that any other annuloplasty device 100 having any other type of annular frame 102 disclosed herein, can include a strip 180 coupled thereto. For example, an elongated strip can be similarly coupled a planar frame 102^{b} that includes a rung of zig-zagging spiked struts 104, or the strip 180 can have a continuous ring-like shape, coupled to an optionally continuous circular planar frame 102, such as any of the planar frame 102 attached to any cylindrical frame 150 disclosed herein.

FIG. 17 shows a side view of an example annuloplasty device 100^{g} that includes a cylindrical frame 150^{g} coupled to an annular frame 102. Annuloplasty device 100^{g} is an example implementation of annuloplasty device 100, and thus can include some or all of the features described for annuloplasty device 100, except that its cylindrical frame 150^{g} can have a tapering shape between the inflow end 152 and the outflow end 154, and the planar frame 102 can be coupled to the inflow end 152 of cylindrical frame 150^{g}. For example, junction 112 of the planar frame 102, such as inner apices 116, can be coupled to the inflow junctions 160 of cylindrical frame 150^{g}.

FIGS. 18A-18C show some phases of an example method of using delivery assembly 200^{g} to implant an annuloplasty device 100^{g}. Delivery apparatus 202^{g} of delivery assembly 200^{g} can be similar to delivery apparatus 202^{e}, comprising a balloon catheter equipped with an inflatable balloon 238. A balloon expandable cylindrical frame 150^{g} of annuloplasty device 100^{g} can be carried in a crimped or radially compressed configuration, over the balloon catheter (including around inflatable balloon 238).

As shown in FIG. 18A, when reaching the site of implantation (for example, mitral valve 36), the deflated balloon 238, carrying crimped cylindrical frame 150^{g} of annuloplasty device 100^{g} thereover, can be advanced to position the planar frame 102 at or in close proximity to the atrial side of annulus 38, with the cylindrical frame 150^{g} oriented distally toward the annulus and/or the left ventricle 34, and can be positioned, in some implementations, between the native leaflets 40.

In this position, as shown in FIG. 18B, the balloon 238 can be inflated to radially expand the cylindrical frame 150^{g} between the native leaflets 40, while the planar frame 102 can engage the atrial side of annulus 38, or improve engagement if it is already in contact with the tissue of the annulus 38. The balloon 238 can be them deflated, and the delivery apparatus 202^{g} can be retrieved from the patient's body, optionally after further constricting the annulus 38 and planar frame 102 by tensioning and locking a tensioning member 138, leaving the annuloplasty device 100^{g} implanted in position, as shown in FIG. 18C.

As mentioned above, the height of any cylindrical frame 150, including when implemented as cylindrical frame 150^{g}, can be kept to a minimum, so as to prevent the cylindrical frame 150^{g} from penetrating too deep through the native annulus towards the ventricle 34. Moreover, the tapering shape of cylindrical frame 150^{g}, which narrows to a smaller diameter in the distal direction, can keep the outflow end 154 radially inward, farther from the leaflets 40, so as to prevent the cylindrical frame 150^{g} from interfering with movement of the native leaflet 40, allowing them to properly coapt in their closed state, as shown in FIG. 18C.

FIGS. 19A and 19B show a side view and a top view, respectively, of an example annuloplasty device 100^{h}. Annuloplasty device 100^{h} is an example implementation of annuloplasty device 100, and in particular, can be similar to annuloplasty device 100^{c} described above with respect to FIGS. 8A-8B, except that the cylindrical frame 150^{h} of annuloplasty device 100^{h} is coupled to the outer apices 114 of the planar frame 102. For example, the outflow junctions 162 at the outflow end 154 of cylindrical frame 150^{h} can be coupled to the outer apices 114 of the planar frame 102.

FIGS. 20A-20C show cross-sectional views of a distal portion of a delivery assembly that includes an example delivery apparatus 202 adapted to deliver and implant annuloplasty device 100^{h}. FIG. 20A shows the annuloplasty device 100^{h} retained entirely inside inner catheter 214, optionally within capsule 218 or otherwise structured distal end portion 216 of inner catheter 214. The cylindrical frame 150^{h} is kept in the radially compressed configuration during delivery inside the inner catheter lumen 220, and is distal to the planar frame 102, which is also retained in a collapsed state during delivery while residing inside the inner catheter 214. As shown, the planar frame 102 is axially aligned with the cylindrical frame 150^{h} when both are kept within inner catheter 214, with the inflow junctions 160 of cylindrical frame 150^{h} being distal to the outflow junction 162.

The cylindrical frame 150^{h} can be formed of a shape-memory material (such as Nitinol), and shape-set to flip over by about 180° when exposed out of the inner catheter lumen 220, as shown in FIG. 20B, such that the inflow junctions 160 of cylindrical frame 150^{h} are positioned proximal to the outflow junction 162. In the configuration shown in FIG. 20B, the annuloplasty device 100^{h} can be partially expelled out of inner catheter 214, for example by utilizing the push member 228 of a push shaft 226 that prevents proximally-oriented movement of the annuloplasty device 100^{h} while the inner catheter 214 is proximally pulled to uncover cylindrical frame 150^{h}, and/or distally moving the push member 228 of push shaft 226 against the planar frame 102 to urge the cylindrical frame 150^{h} out of the inner catheter 214. As the cylindrical frame 150^{h} is freed from inner catheter 214, it flips about outflow junction 162 to assume its pre-shaped orientation, with the inflow junctions 160 being proximal to outflow junctions 162

In some implementations, while the annuloplasty device 100^{h} is only partially expelled out of inner catheter 214, such that the cylindrical frame 150^{h} is freed from the inner catheter 214 and at least a portion of planar frame 102 is still restricted to the collapsed state inside the inner catheter 214, the cylindrical frame 150^{h}, having its outflow junctions 162 attached to the planar frame 102, is forced to remain in a flipped yet compressed configuration thereof. The cylindrical frame 150^{h} can be further shape-set self-expand in a free state thereof.

The planar frame 102 can be formed of a shape-memory material (such as Nitinol), and shape-set to assume a planar state that is substantially orthogonal to a central longitudinal axis Ca of the cylindrical frame 150^{h}, when not bound by an external restricting component. Thus, further deployment of the annuloplasty device 100^{h} out of inner catheter 214, such as by pushing the push shaft 226 and/or pulling the inner catheter 214 to uncover the planar frame 102, will allow the planar frame 102 to spring radially outward to assume its pre-shaped planar state, orthogonal to the cylindrical frame 150^{h}. The free planar frame 102 now also allows for expansion of the cylindrical frame 150^{h}, as shown in FIG. 20C.

FIG. 21 shows the annuloplasty device 100^{h} implanted over a mitral valve 36, following any of the example methods of implantation described above, such that the planar frame 102 is engaged with the atrial side of the annulus 38.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. An annuloplasty device comprising:

a planar frame configured to transition between a collapsed state and an expanded state, the planar frame comprising:

a plurality of spiked struts connected to each other at a plurality of junctions, the plurality of junctions comprising a plurality of outer apices and a plurality of inner apices;

a plurality of expandable segments, wherein each expandable segment comprises at least two of the plurality of spiked struts, and is configured to transition between a collapsed state and an expanded state; and

a plurality of spikes extending from the plurality of spiked struts;

wherein each two spiked struts of the plurality of spiked struts, which are connected to each other at a corresponding junction of the plurality of junctions, are configured to pivotably move relative to each other; and

wherein the plurality of spiked struts and the spikes extending therefrom are coplanar in the expanded state of the planar frame, such that the outer apices are positioned radially away from the inner apices in the expanded state of the planar frame.

Example 2. The annuloplasty device of any example herein, particularly example 1, wherein each expandable segment defines a segmental length between two adjacent junctions of the plurality of junctions comprised in the expandable segment, wherein the two adjacent junctions defining the segmental length are at the same radial level relative to a central frame axis when the expandable segment is in an expanded state thereof.

Example 3. The annuloplasty device of any example herein, particularly example 2, wherein the segmental length of each of the plurality of expandable segments is different between the collapsed state and the expanded state of the expandable segment.

Example 4. The annuloplasty device of any example herein, particularly example 2 or 3, wherein the segmental lengths of all of the expandable segment are identical when all of the expandable segments are in the same state.

Example 5. The annuloplasty device of any example herein, particularly any one of examples 2 to 4, wherein each expandable segment further defines a segmental width which is perpendicular to the segmental length.

Example 6. The annuloplasty device of any example herein, particularly example 5, wherein the segmental width of each of the plurality of expandable segments is different between the collapsed state and the expanded state of the expandable segment.

Example 7. The annuloplasty device of any example herein, particularly example 5 or 6, wherein the segmental widths of all of the expandable segment are identical when all of the expandable segments are in the same state.

Example 8. The annuloplasty device of any example herein, particularly example 5 or 6, wherein the planar frame is further configured to transition to a constricted state.

Example 9. The annuloplasty device of any example herein, particularly example 8, wherein each of the plurality of expandable segments is further configured to transition to a constricted state.

Example 10. The annuloplasty device of any example herein, particularly example 9, wherein the segmental length of each of the plurality of expandable segments is greater in the expanded state than in the constricted state of the expandable segment.

Example 11. The annuloplasty device of any example herein, particularly example 9 or 10, wherein the segmental width of each of the plurality of expandable segments is greater in the constricted state than in the expanded state of the expandable segment.

Example 12. The annuloplasty device of any example herein, particularly any one of examples 9 to 11, further comprising a tensioning member attached to the planar frame at an attachment end portion of the tensioning member.

Example 13. The annuloplasty device of any example herein, particularly example 12, wherein the tensioning member comprises at least one of: a string, a suture, a wire, or a cable.

Example 14. The annuloplasty device of any example herein, particularly example 12 or 13, wherein the attachment end portion comprises loop.

Example 15. The annuloplasty device of any example herein, particularly any one of examples 12 to 14, wherein the attachment end portion comprises knot.

Example 16. The annuloplasty device of any example herein, particularly any one of examples 12 to 15, wherein the attachment end portion is attached to a junction of the plurality of junctions.

Example 17. The annuloplasty device of any example herein, particularly any one of examples 12 to 16, wherein the tensioning member extends from the attachment end portion along junctions of the plurality of junctions that define the plurality of segmental lengths.

Example 18. The annuloplasty device of any example herein, particularly example 17, wherein the tensioning member is coupled to the junctions the define the plurality of segmental lengths.

Example 19. The annuloplasty device of any example herein, particularly example 18, wherein the tensioning member is looped around at least some of the junctions the define the plurality of segmental lengths.

Example 20. The annuloplasty device of any example herein, particularly any one of examples 17 to 19, wherein the tensioning member is slidable along at least some of the junctions the define the plurality of segmental lengths.

Example 21. The annuloplasty device of any example herein, particularly any one of examples 17 to 19, wherein the tensioning member extends through channels formed in at least some of the junctions the define the plurality of segmental lengths.

Example 22. The annuloplasty device of any example herein, particularly any one of examples 17 to 19, wherein the tensioning member extends through eyelets attached to at least some of the junctions the define the plurality of segmental lengths.

Example 23. The annuloplasty device of any example herein, particularly any one of examples 12 to 23, wherein the tensioning member is configured, upon being tensioned, to transition the planar frame to its constricted state.

Example 24. The annuloplasty device of any example herein, particularly any one of examples 12 to 23, wherein the tensioning member is configured, upon being tensioned, to transition at least one expandable segment of the plurality of expandable segments to its constricted state.

Example 25. The annuloplasty device of any example herein, particularly any one of examples 5 to 24, wherein each spiked strut comprises a plurality of the plurality of spikes, extending therefrom.

Example 26. The annuloplasty device of any example herein, particularly any one of examples 5 to 25, wherein each spike of the plurality of spikes terminates at a sharp tip.

Example 27. The annuloplasty device of any example herein, particularly any one of examples 5 to 26, wherein the plurality of spikes is coplanar with the plurality of spiked struts.

Example 28. The annuloplasty device of any example herein, particularly any one of examples 5 to 27, wherein the plurality of spikes is coplanar with the planar frame.

Example 29. The annuloplasty device of any example herein, particularly any one of examples 5 to 28, wherein the spikes extending from each spiked strut of the plurality of spiked struts, extend from a single side of the corresponding spiked strut.

Example 30. The annuloplasty device of any example herein, particularly any one of examples 5 to 29, wherein the plurality of spiked struts form at least one rung of spiked struts.

Example 31. The annuloplasty device of any example herein, particularly example 30, wherein the at least one rung of spiked struts comprises at least two rungs of spiked struts.

Example 32. The annuloplasty device of any example herein, particularly example 31, wherein the at least two rungs of spiked struts define a plurality of spiked cells arranged in at least one row of spiked cells.

Example 33. The annuloplasty device of any example herein, particularly example 32, wherein the at least one row of spiked cells comprises a single row of spiked cells.

Example 34. The annuloplasty device of any example herein, particularly example 32 or 33, wherein each spiked cell of the plurality of spiked cells comprises at least four of the plurality of spiked struts.

Example 35. The annuloplasty device of any example herein, particularly example 34, wherein each spiked cell of the plurality of spiked cells is diamond shaped.

Example 36. The annuloplasty device of any example herein, particularly any one of examples 32 to 35, wherein the plurality of spiked cells are coplanar.

Example 37. The annuloplasty device of any example herein, particularly any one of examples 32 to 36, wherein the spikes extending from each spiked strut of any spiked cells of the plurality of spiked cells, are oriented towards at least one other spiked struts of the same spiked cell.

Example 38. The annuloplasty device of any example herein, particularly any one of examples 32 to 38, wherein the plurality of spiked cells is the plurality of expandable segments.

Example 39. The annuloplasty device of any example herein, particularly example 38, wherein the plurality of junctions further comprise a plurality of intermediate junctions disposed between the plurality of inner apices and the plurality of outer apices.

Example 40. The annuloplasty device of any example herein, particularly example 39, wherein the segmental length of each of the plurality of spiked cells is defined between two opposing intermediate junctions of the plurality of intermediate junctions of the same spiked cell.

Example 41. The annuloplasty device of any example herein, particularly example 39 or 40, wherein the segmental width of each of the plurality of spiked cells is defined between the outer apex and the inner apex of the same spiked cell.

Example 42. The annuloplasty device of any example herein, particularly example 30, wherein the at least one rung of spiked struts comprises a single rung of spiked struts.

Example 43. The annuloplasty device of any example herein, particularly example 42, wherein each of the plurality of expandable segments is defined by two interconnected spiked struts of the plurality of spiked struts.

Example 44. The annuloplasty device of any example herein, particularly example 43, wherein the segmental length of each of the plurality of expandable segments is defined between two adjacent inner apices of the plurality of inner apices of the same expandable segment.

Example 45. The annuloplasty device of any example herein, particularly example 43, wherein the segmental length of each of the plurality of expandable segments is defined between two adjacent outer apices of the plurality of outer apices of the same expandable segment.

Example 46. The annuloplasty device of any example herein, particularly any one of examples 43 to 45, wherein the segmental width of each expandable segment of the plurality of expandable segments is defined as the radial distance between the outer apex and the inner apex of the same expandable segment.

Example 47. The annuloplasty device of any example herein, particularly any one of examples 5 to 46, wherein the planar frame comprises a shape-memory material.

Example 48. The annuloplasty device of any example herein, particularly example 47, wherein the shape-memory material comprises Nitinol.

Example 49. The annuloplasty device of any example herein, particularly any one of examples 5 to 48, wherein the planar frame is a self-expandable frame, configured to assume the expanded state in a free state thereof.

Example 50. The annuloplasty device of any example herein, particularly any one of examples 5 to 49, wherein at least two of the plurality of expandable segments are attached, each, to a single other one of the plurality of expandable segments, while each of the other expandable segments are attached, each, to two the plurality of expandable segments.

Example 51. The annuloplasty device of any example herein, particularly example 50, wherein the plurality of expandable segments comprises a first expandable segment which is coupled to an adjacent one of the expandable segments at one side thereof, and remains free ended without being coupled to any other expandable segment at an opposite side that included a first junction of the plurality of junctions.

Example 52. The annuloplasty device of any example herein, particularly example 50 or 51, wherein the plurality of expandable segments comprises a final expandable segment which is coupled to an adjacent one of the expandable segments at one side thereof, and remains free ended without being coupled to any other expandable segment at an opposite side that included a final junction of the plurality of junctions.

Example 53. The annuloplasty device of any example herein, particularly any one of examples 50 to 52, wherein each two of the expandable segments, attached to each other at a corresponding junction of the plurality of junctions, are angularly movable relative to each other along a plane defined by the planar frame.

Example 54. The annuloplasty device of any example herein, particularly any one of examples 50 to 53, wherein each expandable segment of the plurality of expandable segments is configured to independently transition between its collapsed and expanded states.

Example 55. The annuloplasty device of any example herein, particularly any one of examples 50 to 54, wherein each expandable segment of the plurality of expandable segments is configured to independently self-expand when in a free state thereof.

Example 56. The annuloplasty device of any example herein, particularly any one of examples 5 to 49, further comprising a cylindrical frame coupled to the planar frame, wherein the cylindrical frame is configured to transition between a radially compressed configuration and a radially expanded configuration, and comprises a plurality of angled struts extending between a plurality of inflow junctions at an inflow end of the cylindrical frame, and a plurality of outflow junctions at an outflow end of the cylindrical frame.

Example 57. The annuloplasty device of any example herein, particularly example 56, wherein the plurality of angled struts define a plurality of cells.

Example 58. The annuloplasty device of any example herein, particularly example 57, wherein the plurality of cells define a single row of the cells.

Example 59. The annuloplasty device of any example herein, particularly any one of examples 56 to 58, wherein the planar frame is coupled to the outflow end of the cylindrical frame.

Example 60. The annuloplasty device of any example herein, particularly any one of examples 56 to 59, wherein the plurality of inner apices are coupled to the plurality of outflow junctions.

Example 61. The annuloplasty device of any example herein, particularly example 60, further comprising a plurality of connectors coupling the plurality of inner apices to the plurality of outflow junctions.

Example 62. The annuloplasty device of any example herein, particularly example 61, wherein the plurality of connectors comprises a plurality of suture loops.

Example 63. The annuloplasty device of any example herein, particularly any one of examples 56 to 59, wherein the plurality of outer apices are coupled to the plurality of outflow junctions.

Example 64. The annuloplasty device of any example herein, particularly example 63, wherein the cylindrical frame is configured to flip from a state in which the inflow junctions are distal to the outflow junctions, to a state in which the inflow junctions are proximal to the outflow junctions.

Example 65. The annuloplasty device of any example herein, particularly any one of examples 56 to 58, wherein the planar frame is coupled to the inflow end of the cylindrical frame.

Example 66. The annuloplasty device of any example herein, particularly example 63, wherein the cylindrical frame tapers from the inflow end to the outflow end in its expanded configuration.

Example 67. The annuloplasty device of any example herein, particularly any one of examples 56 to 58, wherein the planar frame and the cylindrical frame are integrally formed.

Example 68. The annuloplasty device of any example herein, particularly example 67, wherein the plurality of inner apices and the plurality of outflow junctions are integrally formed.

Example 69. The annuloplasty device of any example herein, particularly example 67, wherein the plurality of inner apices and the plurality of inflow junctions are integrally formed.

Example 70. The annuloplasty device of any example herein, particularly example 67, wherein the plurality of outer apices and the plurality of outflow junctions are integrally formed.

Example 71. The annuloplasty device of any example herein, particularly any one of examples 56 to 70, wherein the struts of the cylindrical frame are devoid of spikes.

Example 72. The annuloplasty device of any example herein, particularly any one of examples 56 to 71, wherein the planar frame is deflectable relative to the cylindrical frame.

Example 73. The annuloplasty device of any example herein, particularly any one of examples 56 to 72, wherein the planar frame is configured to transition between an axially aligned state and a planar state.

Example 74. The annuloplasty device of any example herein, particularly example 73, wherein the planar frame is in the collapsed state when the cylindrical frame is in the radially compressed configuration.

Example 75. The annuloplasty device of any example herein, particularly example 73 or 74, wherein the planar frame is in the expanded state when the cylindrical frame is in the radially expanded configuration.

Example 76. The annuloplasty device of any example herein, particularly any one of examples 56 to 75, wherein each expandable segment of the plurality of expandable segment is connected, at both sides thereof, to adjacent expandable segments of the plurality of expandable segment.

Example 77. The annuloplasty device of any example herein, particularly any one of examples 56 to 62, wherein the cylindrical frame further comprises a plurality of engaging struts extending proximally from the plurality of inflow junctions.

Example 78. The annuloplasty device of any example herein, particularly example 77, wherein each of the plurality of engaging struts comprises a tab at a proximal end thereof.

Example 79. The annuloplasty device of any example herein, particularly any one of examples 1 to 78, further comprising a strip coupled to the planar frame, the strip comprising a fabric base layer and a plurality of floating yarns extending therefrom.

Example 80. A method comprising:

navigating a delivery assembly that comprises an annuloplasty device having a planar frame, towards a native valve, wherein the planar frame is retained in a collapsed state thereof inside an inner catheter of a delivery apparatus of the delivery assembly;

deploying the planar frame out of the inner catheter and engaging spiked struts of the planar frame with an annulus of the native valve, such that the spiked struts and spikes extending therefrom are coplanar with a plane defined by the annulus; and

plicating tissue of annulus between adjacent spiked struts of a plurality of spiked struts of the planar frame.

Example 81. The method of any example herein, particularly example 80, wherein the navigating the delivery assembly comprises steering the inner catheter towards the native valve.

Example 82. The method of any example herein, particularly example 80 or 81, wherein each expandable segment of the plurality of expandable segments defines a segmental length between two adjacent junctions of the expandable segment, wherein the two adjacent junctions defining the segmental length are at the same radial level relative to a central frame axis when the expandable segment is in an expanded state thereof.

Example 83. The method of any example herein, particularly example 82, wherein each expandable segment of the plurality of expandable segments defines a segmental width which is perpendicular to the segmental length.

Example 84. The method of any example herein, particularly example 82 or 83, wherein each of the expandable segments comprises a spiked cell defined by at least four spiked struts of the plurality of spiked struts.

Example 85. The method of any example herein, particularly example 82 or 83, wherein each of the expandable segments comprises at least two interconnected spiked struts of the plurality of spiked struts.

Example 86. The method of any example herein, particularly any one of examples 82 to 85, wherein at least two of the plurality of expandable segments are attached, each, to a single other one of the plurality of expandable segments, while each of the other expandable segments are attached, each, to two the plurality of expandable segments.

Example 87. The method of any example herein, particularly example 86, wherein the navigating the delivery assembly comprises retaining the plurality of expandable segments, in collapsed states thereof inside the inner catheter, such that their segmental lengths are oriented parallel to a longitudinal axis of the inner catheter.

Example 88. The method of any example herein, particularly example 86 or 87, wherein the navigating the delivery assembly comprises steering the inner catheter towards the annulus.

Example 89. The method of any example herein, particularly any one of examples 86 to 88, wherein the deploying the planar frame comprises sequentially deploying the expandable segments out of the inner catheter, over a curved path along the annulus.

Example 90. The method of any example herein, particularly example 89, wherein each two interconnected expandable segments of the plurality of expandable segments are angularly movable relative to each other, and wherein the deploying the expandable segments over a curved path comprises extending the expandable segments such that their segmental lengths are angularly oriented relative to each other.

Example 91. The method of any example herein, particularly any one of examples 86 to 90, wherein the deploying the planar frame comprises uncovering the expandable segments by retracting the inner catheter.

Example 92. The method of any example herein, particularly any one of examples 86 to 91, wherein the deploying the planar frame comprises uncovering the expandable segments by distally pushing a deployment plug attached to a deployment shaft of the delivery assembly, against the planar frame.

Example 93. The method of any example herein, particularly any one of examples 86 to 92, wherein the deploying the planar frame out of the inner catheter further comprises expanding each of the plurality of expandable segments which is uncovered from the inner catheter.

Example 94. The method of any example herein, particularly example 93, wherein the planar frame comprises a shape-memory material, and wherein each of the expandable segments is configured to self-expand in a free state thereof.

Example 95. The method of any example herein, particularly example 93 or 94, wherein the deploying the planar frame comprises placing each of the expandable segments, uncovered from the inner catheter, over the annulus such that the spikes engage with the annulus.

Example 96. The method of any example herein, particularly any one of examples 93 to 95, wherein the expanding each of the plurality of expandable segments comprises approximating the junctions defining the segmental length of the corresponding expandable segment, thereby shortening the segmental length relative to the collapsed state of the same expandable segment.

Example 97. The method of any example herein, particularly any one of examples 93 to 96, wherein the plicating the tissue of the annulus comprises the expanding the expandable segments.

Example 98. The method of any example herein, particularly any one of examples 93 to 96, wherein the plicating the tissue of the annulus comprises proximally pulling a tensioning member attached to the planar frame at an attachment end portion of the tensioning member.

Example 99. The method of any example herein, particularly example 98, wherein the attachment end portion is coupled to a first junction of the planar frame, wherein the tensioning member extends from the first junction along the remaining junctions defining the segmental lengths towards a final junction of the planar frame, and extends proximally from the final junction.

Example 100. The method of any example herein, particularly example 99, wherein the tensioning member extends and is slidably movable through channels formed within at least some of the junctions defining the segmental lengths.

Example 101. The method of any example herein, particularly example 99, wherein the tensioning member extends and is slidably movable through eyelets of at least some of the junctions defining the segmental lengths.

Example 102. The method of any example herein, particularly any one of examples 98 to 101, wherein the pulling the tensioning member comprises transitioning at least one of the expandable segments to a constricted state, wherein the segmental length of the corresponding at least one expandable segment in the constricted state is less than the segmental length in its expanded state.

Example 103. The method of any example herein, particularly any one of examples 98 to 102, further comprising locking the tensioning member by a locker.

Example 104. The method of any example herein, particularly example 103, wherein the locking the tensioning member further comprises trimming the tensioning member.

Example 105. The method of any example herein, particularly any one of examples 82 to 85, wherein each of the plurality of expandable segments is disposed between and attached to another two of the plurality of expandable segments.

Example 106. The method of any example herein, particularly example 105, wherein the annuloplasty device further comprises a cylindrical frame coupled to the planar frame, wherein the cylindrical frame is configured to transition between a radially compressed configuration and a radially expanded configuration.

Example 107. The method of any example herein, particularly example 106, wherein the planar frame is coupled to an outflow end of the cylindrical frame.

Example 108. The method of any example herein, particularly example 106 or 107, wherein a plurality of inner apices of the planar frame are coupled to a plurality of outflow junctions of the cylindrical frame.

Example 109. The method of any example herein, particularly example 108, wherein the annuloplasty device further comprises a plurality of connectors coupling the plurality of inner apices to the plurality of outflow junctions.

Example 110. The method of any example herein, particularly example 106 or 107, wherein a plurality of outer apices of the planar frame are coupled to a plurality of outflow junctions of the cylindrical frame.

Example 111. The method of any example herein, particularly example 110, wherein the annuloplasty device further comprises a plurality of connectors coupling the plurality of outer apices to the plurality of outflow junctions.

Example 112. The method of any example herein, particularly example 110 or 111, wherein the cylindrical frame is configured to flip from a state in which the inflow junctions are distal to the outflow junctions, to a state in which the inflow junctions are proximal to the outflow junctions.

Example 113. The method of any example herein, particularly example 109 or 111, herein the plurality of connectors comprises a plurality of suture loops.

Example 114. The method of any example herein, particularly example 106 or 107, wherein the planar frame and the cylindrical frame are integrally formed.

Example 115. The method of any example herein, particularly any one of examples 106 or 107, wherein the plurality of inner apices and the plurality of outflow junctions are integrally formed.

Example 116. The method of any example herein, particularly any one of examples 110 or 111, wherein the plurality of inner apices and the plurality of outflow junctions are integrally formed.

Example 117. The method of any example herein, particularly any one of examples 106 to 115, wherein the planar frame is configured to transition between an axially aligned state and a planar state.

Example 118. The method of any example herein, particularly example 117, wherein the navigating the delivery assembly comprises retaining the cylindrical frame in the radially compressed configuration and retaining the planar frame in the aligned state inside the inner catheter.

Example 119. The method of any example herein, particularly example 118, wherein the deploying the planar frame comprises at least partially expelling the annuloplasty device from the inner catheter such that the planar frame transitions to the planar state, while the cylindrical frame remains in the radially compressed configuration.

Example 120. The method of any example herein, particularly example 119, wherein the planar frame remains in the collapsed state while the cylindrical frame remains in the radially compressed configuration.

Example 121. The method of any example herein, particularly example 119 or 120, wherein the at least partially expelling the annuloplasty device comprises retracting the inner catheter from at least a portion of the annuloplasty device.

Example 122. The method of any example herein, particularly any one of examples 119 to 121, wherein the at least partially expelling the annuloplasty device comprises distally pushing a deployment plug attached to a deployment shaft of the delivery apparatus, against the annuloplasty device.

Example 123. The method of any example herein, particularly example 118, wherein the navigating the delivery assembly comprises retaining the inflow junctions distal to the outflow junctions inside the inner catheter.

Example 124. The method of any example herein, particularly example 123, wherein the deploying the planar frame comprises fully expelling the annuloplasty device from the inner catheter such that the cylindrical frame is flipped to position the inflow apices proximal to the outflow apices, and the planar frame transitions to the planar state.

Example 125. The method of any example herein, particularly any one of examples 119 to 121, wherein the deploying the planar frame further comprises positioning the planar frame, in its planar state, over the annulus, such that the spikes engage with the annulus.

Example 126. The method of any example herein, particularly example 125, wherein the positioning the planar frame over the annulus comprises positioning the cylindrical frame in an atrium at one side of the native valve.

Example 127. The method of any example herein, particularly any one of examples 119 to 122, wherein the at least partially expelling the annuloplasty device comprises retaining the cylindrical frame inside the inner catheter.

Example 128. The method of any example herein, particularly example 127, wherein the deploying the planar frame further comprises expanding the cylindrical frame by uncovering the cylindrical frame from the inner catheter, thereby transitioning the planar frame to the expanded state.

Example 129. The method of any example herein, particularly example 124, wherein the deploying the planar frame further comprises transitioning the planar frame to the expanded state by expanding the cylindrical frame.

Example 130. The method of any example herein, particularly example 128 or 129, wherein the cylindrical frame is made of a shape memory material and wherein the expanding the cylindrical frame comprises allowing the cylindrical frame to self-expand.

Example 131. The method of any example herein, particularly any one of examples 119 to 122, wherein the at least partially expelling the annuloplasty device comprises deploying the annuloplasty device out of the inner catheter, while the cylindrical frame is disposed in the radially compressed configuration, around a deflated inflatable balloon mounted on a balloon catheter of the delivery apparatus.

Example 132. The method of any example herein, particularly example 106, wherein the planar frame is coupled to an inflow end of the cylindrical frame.

Example 133. The method of any example herein, particularly example 132, wherein the cylindrical frame tapers from the inflow end to the outflow end in its expanded configuration.

Example 134. The method of any example herein, particularly example 132 or 133, wherein the deploying the planar frame further comprises deploying the annuloplasty device out of the inner catheter, while the cylindrical frame is disposed in the radially compressed configuration, around a deflated inflatable balloon mounted on a balloon catheter of the delivery apparatus, and extends distally from the planar frame.

Example 135. The method of any example herein, particularly example 134, wherein the deploying the annuloplasty device out of the inner catheter comprises positioning the cylindrical frame between native leaflets of the native valve.

Example 136. The method of any example herein, particularly example 131 or 134, wherein the deploying the planar frame further comprises expanding the cylindrical frame by inflating the inflatable balloon, thereby transitioning the planar frame to the expanded state.

Example 137. The method of any example herein, particularly example 136, further comprises, subsequent to the expanding the cylindrical frame, deflating the inflatable balloon.

Example 138. The method of any example herein, particularly example 128 or 136, wherein the transitioning the planar frame to the expanded state comprises distancing the junctions defining the segmental lengths of the corresponding expandable segments, thereby elongating the segmental lengths relative to the collapsed state of the planar frame.

Example 139. The method of any example herein, particularly any one of examples 128 to 138, wherein the plicating the tissue of the annulus comprises proximally pulling a tensioning member attached to the planar frame at an attachment end portion of the tensioning member.

Example 140. The method of any example herein, particularly example 139, wherein the attachment end portion is coupled to one of the junctions of the planar frame, wherein the tensioning member extends from the attachment end portion along a circular path passing through the remaining junctions defining the segmental lengths, back towards the attachment end portion, and proximally therefrom.

Example 141. The method of any example herein, particularly example 140, wherein the tensioning member extends and is slidably movable through channels formed within at least some of the junctions defining the segmental lengths.

Example 142. The method of any example herein, particularly example 140, wherein the tensioning member extends and is slidably movable through eyelets of at least some of the junctions defining the segmental lengths.

Example 143. The method of any example herein, particularly any one of examples 139 to 142, wherein the pulling the tensioning member comprises transitioning the planar frame to a constricted state, wherein the segmental lengths in the constricted state are shorter than the segmental lengths in its expanded state of the planar frame.

Example 144. The method of any example herein, particularly any one of examples 139 to 143, further comprising locking the tensioning member by a locker.

Example 145. The method of any example herein, particularly example 144, wherein the locking the tensioning member further comprises trimming the tensioning member.

Example 146. The method of any example herein, particularly any one of examples 80 to 145, further comprising, subsequent to the plicating the tissue of the annulus, retracting the delivery apparatus.

Example 147. The method of any example herein, particularly any one of examples 80 to 146, wherein the native valve is a mitral valve.

Example 148. The method of any example herein, particularly any one of examples 80 to 146, wherein the native valve is a tricuspid valve.

Example 149. The method of any example herein, particularly any one of examples 80 to 146, wherein the annuloplasty device further comprises a strip coupled to the planar frame, the strip comprising a fabric base layer and a plurality of floating yarns extending therefrom towards the annulus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. Apparatus, comprising an implant for use at a valve of a heart of a subject, the implant:
having a central axis, and
comprising a frame that defines a row of spiked cells arranged in a ring around the central axis, each of the spiked cells having four corners comprising:
an inner apex,
two lateral corners, each of which is connected circumferentially, at a respective intermediate junction, to a lateral corner of a respective adjacent cell of the row,
an outer apex, oriented away from the central axis,
wherein, for each of the spiked cells of the row, the circumferential connections at the intermediate junctions facilitate pivoting of the outer apex toward and away from the central axis independently of the outer apices of the adjacent cells in a manner that maintains spikes of the cell in tissue-gripping contact with tissue of the valve.

2. The apparatus according to claim 1, wherein each cell of the row is defined by struts that connect the four corners, the cell defines a space between the struts, and the spikes are disposed on the struts.

3. The apparatus according to claim 2, wherein the spikes extend from the struts in a manner co-planar with a plane defined by the cell.

4. The apparatus according to claim 3, wherein, for each strut of each spiked cell, the spikes extend only from one side of the strut, an opposite side of the strut being devoid of spikes.

5. The apparatus according to claim 4, wherein the implant is deliverable to the heart while compressed within a catheter, and the frame is configured such that, while the implant is compressed within the catheter, the opposite side of each struts faces an inner wall of the catheter.

6. The apparatus according to claim 3, wherein, for each cell, the spikes extend into the space to engage a portion of the tissue that protrudes into the space.

7. The apparatus according to claim 3, wherein, for each cell, the spikes extend toward each other.

8. The apparatus according to claim 1, wherein the implant has a compressed delivery state, and is configured to self-expand upon deployment within the heart, the self-expansion including a change in distance between the intermediate junctions.

9. The apparatus according to claim 1, wherein the frame is dimensioned such that all of the outer apices are equidistant from the central axis.

10. The apparatus according to claim 1, wherein the frame is a first frame, the implant further comprises a cylindrical frame coupled to the first frame, and the spiked cells extend radially away from the cylindrical frame such that the outer apices are the most radially distant part of the spiked cells from the cylindrical frame.

11. The apparatus according to claim 10, wherein the cylindrical frame comprises a plurality of angled struts defining a single row of cells.

12. The apparatus according to claim 10, wherein the cylindrical frame is balloon-expandable.

13. The apparatus according to claim 10, wherein the cylindrical frame is self-expandable and formed of a shape-memory material.

14. The apparatus according to claim 10, wherein the cylindrical frame further comprises a plurality of tabs at inflow junctions, the tabs configured for releasable coupling to a delivery tool.

15. The apparatus according to claim 10, wherein an inflow end of the cylindrical frame is disposed axially upstream of the outer apices.

16. The apparatus according to claim 10, wherein an inflow end of the cylindrical frame is disposed axially upstream of the ring.

17. An apparatus comprising an implant for use at a valve of a heart of a subject, the implant comprising:
a central axis, and
a frame that defines a row of spiked cells arranged in a ring around the central axis, each of the cells having four corners comprising:
an inner apex,
two lateral corners, each of which is connected circumferentially, at a respective intermediate junction, to a lateral corner of a respective adjacent cell of the row,
an outer apex, oriented away from the central axis,
wherein the circumferential connections at the intermediate junctions facilitate bending of each spiked cell with respect to the adjacent cells such that its outer apex moves toward and away from the central axis independently of the outer apices of the adjacent cells in a manner that maintains spikes of the cell in tissue-gripping contact with tissue of the valve.

18. The apparatus according to claim 17, wherein the frame is a first frame, the implant further comprises a cylindrical frame coupled to the first frame, and the spiked cells extend radially away from the cylindrical frame such that the outer apices are the most radially distant part of the spiked cells from the cylindrical frame.

19. An apparatus comprising an implant for use at a native cardiac valve of a subject, the implant comprising:
a central axis; and
a frame that, in an implanted configuration, defines a circumferential ring of spiked cells around the central axis, each spiked cell comprising:
four corners comprising:
an inner apex,
two lateral corners connected, at respective intermediate junctions, to lateral corners of adjacent cells, and
an outer apex oriented radially away from the central axis,
struts that carry spikes,
wherein the intermediate junctions provide circumferential articulation such that each spiked cell bends with respect to adjacent cells to move its outer apex toward and away from the central axis independently of outer apices of the adjacent cells in a manner that maintains the spikes of that cell in tissue-gripping contact with tissue of the cardiac valve.

20. The apparatus according to claim 19, wherein the frame is a first frame, the implant further comprises a cylindrical frame coupled to the first frame, and the spiked cells extend radially away from the cylindrical frame such that the outer apices are the most radially distant part of the spiked cells from the cylindrical frame.
